(19) United States
(12) Patent Application Publication
(10) Pub. No.: US 2007/0264222 A1
(43) Pub. Date: Nov. 15, 2007

(54) ENHANCED PROTECTION AGAINST SKIN INJURY IN HUMANS

(75) Inventor: Izolda M. Georgiades, Houston, TX (US)

Correspondence Address: CHALKER FLORES, LLP, 2711 LBJ FRWY, Suite 1036, DALLAS, TX, 75234, US

(73) Assignee: BIOHEALTH ADVANCE, LLC, Highland Village, TX (US)

(21) Appl. No.: 11/746,733

(22) Filed: May 10, 2007

Related U.S. Application Data
(60) Provisional application No. 60/799,184, filed on May 10, 2006.
(60) Provisional application No. 60/831,592, filed on Jul. 19, 2006.

(51) Int. Cl. A61K 8/97; A61K 31/22; A61K 31/202; A61K 36/72

(52) U.S. Cl. 424/74; 424/777; 514/547; 514/560

(57) ABSTRACT

The present invention includes compositions and methods for treating skin conditions, and more particularly, a medicinal progression of anti-oxy-vitamins to treat one or more skin conditions in a patient selected from at least one of acne, skin discoloration, comedo presence, foci of inflammation, hyper-keratinosis, enlarged sebum vessels, pores enlargement, scars or piling centers.

### REFERENCE TO RELATED APPLICATIONS

This application claims priority to U.S. Provisional Patent Application No. 60/799,184 filed, May 10, 2006, and U.S. Provisional Patent Application No. 60/831,592, filed, Jun. 19, 2006, the entire contents of each of which are incorporated herein by reference.

### TECHNICAL FIELD OF THE INVENTION

The present invention relates in general to the field of dietary supplementation, and more particularly, to compositions and methods that enhanced protection against skin injury in humans.

### BACKGROUND OF THE INVENTION

Without limiting the scope of the invention, its background is described in connection with supplements that help protect the body from oxidative stress. The significance of the oxidative stress-induced injury to skin has been the subject of several reviews. Also many inventions relates to various protocols for treatment of many skin conditions as outlined in the following patents.

U.S. Pat. No. 4,342,747 discloses colostrums-based compositions for external ophthalmic use as a therapeutic agent and/or as a there substitute. U.S. Pat. No. 5,698,185 discloses a whitening cosmetic preparation containing angiogenin as an effective component, the angiogenin may be derived from milk, especially bovine milk. U.S. Pat. No. 5,750,149 discloses pharmaceutical and/or dermo-cosmetic compositions containing equine colostrums, optionally in ad mixture with milk. U.S. Pat. No. 6,187,297 discloses a composition that includes Sea Buckthorn Oil as a cosmetic product, a pharmaceutical or nutraceutical product or a food product. The food product can be used in combination with the cosmetic applications of the present invention. U.S. Pat. No. 6,221,918 discloses combinations of carotenoids for use as a dietary supplement or topical ointment.

U.S. Pat. No. 6,258,383 discloses a dietary supplement comprising colostrums, lactoferrin, and with modified citrus pectin as an optional component, administered in ‘mucosal delivery format’: a dosage form that promotes effective absorption through the lining of the oral cavity. U.S. Pat. No. 6,319,522 discloses pharmaceutical or veterinary compositions for the treatment of surface wounds; pharmaceutical or veterinary compositions for the treatment of gastrointestinal injuries, diseases or ulcers; methods of treating surface wounds in animals, including humans; and methods for the treatment of gastrointestinal injuries, diseases or ulcers which compositions and methods include compositions of milk product extracts including growth factors with basic to approximately neutral isoelectric points. U.S. Pat. No. 6,410,058 discloses a dietary supplement comprising colostrums, lactoferrin, and with modified citrus pectin as an optional component, administered in ‘mucosal delivery format’: a dosage form that promotes effective absorption through the lining of the oral cavity.

U.S. Pat. No. 6,447,808 discloses pharmaceutical or veterinary compositions for the treatment of surface wounds; pharmaceutical or veterinary compositions for the treatment of gastrointestinal injuries, diseases or ulcers; methods of treating surface wounds in animals, including humans; and methods for the treatment of gastrointestinal injuries, diseases or ulcers which compositions and methods include compositions of milk product extracts including growth factors with basic to approximately neutral isoelectric points. U.S. Pat. No. 6,475,511 discloses a dietary supplement comprising colostrums, lactoferrin, and with modified citrus pectin as an optional component, administered in ‘mucosal delivery format’: a dosage form that promotes effective absorption through the lining of the oral cavity. U.S. Pat. No. 6,479,627 discloses an aqueous preparation of lactoferrin where stability of lactoferrin in an aqueous solution is improved and also to a method of stabilizing lactoferrin in an aqueous solution.

U.S. Patent Application No. 2001/0000466 discloses a composition that includes Sea Buckthorn Oil as a cosmetic product, a pharmaceutical or nutraceutical product or a food product. U.S. Patent Application No. 2001/0009681 discloses a dietary supplement comprising colostrums, lactoferrin, and with modified citrus pectin as an optional component, administered in ‘mucosal delivery format’: a dosage form that promotes effective absorption through the lining of the oral cavity. U.S. Patent Application No. 2002/0001625 discloses pharmaceutical or veterinary compositions for the treatment of surface wounds; pharmaceutical or veterinary compositions for the treatment of gastrointestinal injuries, diseases or ulcers; methods of treating surface wounds in animals, including humans; and methods for the treatment of gastrointestinal injuries, diseases or ulcers which compositions and methods include compositions of milk product extracts including growth factors with basic to approximately neutral isoelectric points. U.S. Patent Application No. 2002/0054917 discloses dietary supplement compositions containing beta glucan and lactoferrin, formulated as a liquid, tablet, lozenge, gum or other acceptable form. However, no disclosure has been made regarding specific composition of biologically active substances given concomitantly orally and topically.

### SUMMARY OF THE INVENTION

The present invention relates generally to a novel administration of various biologically active substances in cosmetics. When combined, and/or administered concomitantly, these substances provide the whole human body with the specific biological ingredients, needed at given time, to prevent and treat dermatological conditions or disorders and slowing the aging processes in human bodies through control of oxidative processes. More specifically, the present invention is directed toward the administration of metallo-protein with vitamin E, pro-vitamins A vitamins H, B, C, D, E, whey peptides and Antocyanes a potent anti free radicals scavengers. Those biologically active compounds are mixed in creams and body lotion for a topical application and concomitantly given orally in form of dietary supplements. Since during the life spun production of free radicals gradually increase, the host require stronger anti oxidant complexes. Biovien® creams 1-7 were constructed to cover whole life spun of human being. In construction of Biovien series of cream, body lotion, and dietary supplements, the reverse pyramid principles was applied. To achieve amplification reaction of the body we recommended use simultaneously. Topical application of creams and body lotion with oral application of Biovien dietary supplement.

In one aspect, the present invention is a medicinal composition to prevent or control the effects of free radicals on skin that includes a composition comprising a metalloprotein and fatty acids in an amount sufficient to improve the appearance of skin; and optionally comprising one or more of the following: (a) conjugated linoleic acids and vitamin E; (b) conjugated linoleic acids, plant-derived lipids, Hippohae rhamnoides L. berry extract (“oblepicha”) and vitamin E; (c) conjugated linoleic acids, plant-derived lipid, Hippohae rhamnoides L. berry extract, omega fatty acids and vitamin E; (d) conjugated linoleic acids, plant-derived lipid, Hippohae rhamnoides L. berry extract, omega fatty acids, low molecular weight whey protein extract and vitamin E; (e) conjugated linoleic acids, plant-derived lipid, Hippohae rhamnoides L. berry extract, omega fatty acids, low molecular weight whey protein extract and vitamin E; and (f) conjugated linoleic acids, plant-derived lipid, Hippohae rhamnoides L. berry extract, omega fatty acids, low molecular weight whey protein extract, vitamin E and Antocyanes, wherein the addition of (a) to (f) is dependent on the extent of free-radical damage to the skin, wherein (a) is used to treat a lower level of skin damage and (f) is used to treat the highest levels of free-radical damage to skin, with (b) to (e) added progressively as skin damage increases, respectively.

In one embodiment, an equivalent oral equivalent of the composition is provided to the user at the same time. Another embodiment is the use of an oral equivalent of the composition and a cream equivalent of the composition. The composition of the present invention improves the appearance of skin is measured by decreased pH, inflammation, comedon, acne, hyperkeratosis, enlargement of sebum vessels, pores enlargement, piling centers, skin discoloration or scars. In another embodiment, the composition includes one or more anti bacterial agents. In another embodiment, the metalloprotein comprises ferritin or lactoferrin. In another embodiment, the composition is non-toxic. In another embodiment, the composition is non-allergenic. In another embodiment, the composition further comprises one or more anti bacterial agents. In another embodiment, the low molecular weight whey protein extract comprises small molecular weight peptides from whey fraction from 3 to 14 kilodaltons.

The present invention also includes a method for preventing and treating the effects of free-radicals using a medicinal progression of anti-oxy-vitamins by treating the skin of a subject based on the extent of free-radical damage to the skin, wherein a first, second, third, fourth, fifth, sixth or seventh composition is selected based on an increased level of severity of the free-radical damage to the skin in ascending order, respectively, the compositions from: a first composition comprising lactoferrin and fatty acids in an amount sufficient to decrease oily secretions from skin; a second composition comprising a metalloprotein, fatty acids, conjugated linoleic acids and vitamin E; a third composition comprising a metalloprotein, fatty acids, conjugated linoleic acids, Hippohae rhamnoides L. berry extract (“oblepicha”) and vitamin E; a fourth composition comprising a metalloprotein, fatty acids, Hippohae rhamnoides L. berry extract, omega fatty acid and vitamin E; a fifth composition comprising a metalloprotein, conjugated linoleic acids, Hippohae rhamnoides L. berry extract, omega fatty acids, low molecular weight whey protein extract and vitamin E; a sixth composition comprising a metalloprotein, conjugated linoleic acids, Hippohae rhamnoides L. berry extract, omega fatty acids, low molecular weight whey protein extract and vitamin E; and a seventh composition comprising a metalloprotein, conjugated linoleic acids, Hippohae rhamnoides L. berry extract, omega fatty acids, low molecular weight whey protein extract, vitamin E and antocyanes, wherein a patient uses one or more of the first to seventh serums based on the condition of their skin. In another embodiment, the low molecular weight whey protein extract comprises small molecular weight peptides from whey fraction from 3 to 14 kilodaltons. In another embodiment, the effects of free-radicals on skin is measured by at least one of decreased pH, inflammation, comedon, acne, hyperkeratosis, enlargement of sebum vessels, pores enlargement, piling centers, skin discoloration or scars. In another aspect, the present invention is a composition that includes a metallo-protein, vitamin E, pro-vitamin A, vitamin H, vitamin B, vitamin C, vitamin D, vitamin E, low molecular weight whey protein extract and antocyanes, wherein the composition had a greater free radical scavenging effect than the individual agents alone.

In another aspect, the present invention is a composition for reducing the pH of skin with Lactoferrin and conjugated linoleic acids adapted for topical administration, each of which is provided in an amount sufficient and to lower the pH of skin. The composition may further comprise at least one of a Hippohae rhamnoides L. berry extract, Flax oil, anthocynaes and Vitamin E.

In another aspect, the present invention includes a medicinal progression of anti-oxidants that includes a first composition comprising lactoferrin and fatty acids; a second composition comprising lactoferrin, fatty acids, conjugated linoleic acids and vitamin E; a third composition comprising lactoferrin, fatty acids, conjugated linoleic acids, Hippohae rhamnoides L. berry extract (“oblepicha”) and vitamin E; a fourth composition comprising lactoferrin, fatty acids, Hippohae rhamnoides L. berry extract, omega fatty acid and vitamin E; a fifth composition comprising lactoferrin, conjugated linoleic acids, Hippohae rhamnoides L. berry extract, omega fatty acids, low molecular weight whey protein extract and vitamin E; a sixth composition comprising lactoferrin, conjugated linoleic acids, Hippohae rhamnoides L. berry extract, omega fatty acids, low molecular weight whey protein extract and vitamin E; and a seventh composition comprising lactoferrin, conjugated linoleic acids, Hippohae rhamnoides L. berry extract, omega fatty acids, low molecular weight whey protein extract, vitamin E and antocyanes, wherein a patient uses one or more of the first to seventh serums based on the condition of their skin.

In another aspect, the present invention includes a method of treating one or more skin conditions by evaluating one or more skin conditions in a patient selected from at least one of decreased pH, inflammation, comedon, acne, hyperkeratosis, enlargement of sebum vessels, pores enlargement, piling centers, skin discoloration or scars; and selecting one or more of the following serums that best treats the one or more skin conditions selected from: a first composition comprising lactoferrin and fatty acids; a second composition comprising lactoferrin, fatty acids, conjugated linoleic acids and vitamin E; a third composition comprising lactoferrin, fatty acids, conjugated linoleic acids, Hippohae rhamnoides L. berry extract (“oblepicha”) and vitamin E; a fourth serum comprising lactoferrin, fatty acids, Hippohae rhamnoides L. berry extract, omega fatty acid and vitamin E; a fifth composition comprising lactoferrin, conjugated linoleic acids, Hippohae rhamnoides L. berry extract, omega fatty acids, low molecular weight whey protein extract and vitamin E; a sixth composition comprising lactoferrin, conjugated linoleic acids, Hippohae rhamnoides L. berry extract, omega fatty acids, low molecular weight whey protein extract and vitamin E; and a seventh composition comprising lactoferrin, conjugated linoleic acids, Hippohae rhamnoides L. berry extract, omega fatty acids, low molecular weight whey protein extract, vitamin E and antocyanes, wherein a patient uses one or more of the first to seventh serums based on the condition of their skin.

In another aspect, the present invention includes a method reducing the pH of skin by identifying a subject in need of skin pH correction; and applying to the subject a composition comprising Lactoferrin and conjugated linoleic acids adapted for topical administration, each of which is provided in an amount sufficient and to lower the pH of skin. In one embodiment, the composition further comprises at least one of a Hippohae rhamnoides L. berry extract, Flax oil, anthocynaes and Vitamin E. In another embodiment, the subject is further provided with an equivalent oral equivalent of the composition. In another embodiment, the subject is further provided with an oral equivalent of the composition and a cream equivalent of the composition. In another embodiment, the composition further comprises one or more anti bacterial agents. In another embodiment, the composition is non-toxic, non-allergenic or both. In another embodiment, the composition comprises lactoferrin, fatty acids, conjugated linoleic acids, Hippohae rhamnoides L. berry extract (“oblepicha”) controls aging as measured by at least one of reduced decreased pH, inflammation, comedon, acne, hyperkeratosis, enlargement of sebum vessels, pores enlargement, piling centers, skin discoloration or scars.

In another aspect, the present invention includes a method for reducing the pH of skin comprising: identifying the needs of a subject for skin treatment by evaluating at least one of decreased pH, inflammation, comedon, acne, hyperkeratosis, enlargement of sebum vessels, pores enlargement, piling centers, skin discoloration or scars; matching the needs of the subject with an anti-oxy-vitamin composition adapted for topical administration comprising Lactoferrin, conjugated linoleic acids and one or more of the following agents: Hippohae rhamnoides L. berry extract, Flax oil, anthocynaes and Vitamin E; and applying to the subject the composition in an amount sufficient and to treat the at least one of inflammation, comedon, acne, hyperkeratosis, enlargement of sebum vessels, pores enlargement, piling centers, skin discoloration or scars. In one embodiment, use of the composition slows down the appearance of aging as measured by at least one of reduced skin wrinkles, increases in skin elasticity, reduced skin discoloration, reduced scar formation, reduced sebum production, increased hydration effect and decreased skin pH. In another embodiment, the patient concomitantly with an equivalent of the composition for skin treatment in a form adapted for oral administration.

In yet another aspect, the present invention includes a method for reversing the damage to skin caused by free-radicals by applying to the skin of a subject with skin damage with an effective amount of an anti-oxy-vitamin composition adapted for topical administration comprising Lactoferrin, conjugated linoleic acids and one or more of the following agents: Hippohae rhamnoides L. berry extract, Flax oil, anthocynaes and Vitamin E, wherein the skin damage comprises at least one of decreased pH, inflammation, comedon, acne, hyperkeratosis, enlargement of sebum vessels, pores enlargement, piling centers, skin discoloration or scars.

### BRIEF DESCRIPTION OF THE DRAWINGS

For a more complete understanding of the features and advantages of the present invention, reference is now made to the detailed description of the invention along with the accompanying figures and in which:

FIG. 1 is a graph that demonstrates the changes in pH of the volunteer's skin treated with Biovien® 1 body lotion.

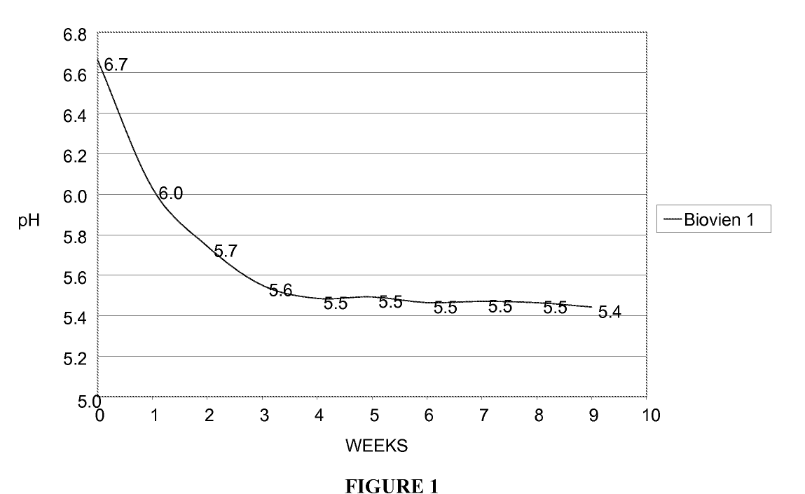

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

As used herein, the term “anti-oxy-vitamin” refers to any molecule that will bind free radicals of different origin will, among them are: polifenoles, flavonoids carotens, retinoids, antocyane. Anti-oxy-vitamins are not synthesized by human body and need to be delivered with other food or isolated from food compounds. Some of them represent a quite large family of similar compounds, other are classified as pro-vitamins and other are considered as mono antyoxidants. Comon future is the protection of human organism, especially the skin, against UV light.

As used herein, the term “free radicals” refers to free radicals are ions or molecules, which at their external orbit have unpaired electron. Free radicals include all intermediates, inanabolic, and catabolic pathways when they escape.

As used herein, the terms “healthy skin” or “normal skin” refer to non-lesional skin, i.e., with no visually obvious erythema, edema, hyper-, hypo-, or uneven pigmentations, scale formation, xerosis, or blister formation. Skin that is histologically healthy or normal refers to skin tissue with a morphological appearance of well-organized basal, spinous, and granular layers, and a coherent multi-layered stratum corneum. Normal or healthy epidermis may include terminally differentiated, stratified squamous epithelium with an undulating junction with the underlying dermal tissue. Normal or healthy skin may also be determined by a lack of fluid retention, cellular infiltration, hyper- or hypoproliferation of any cell types, mast cell degranulation, parakeratoses and normal levels of Langerhans cells and dermal dendrocytes. The skilled artisan will recognize healthy or normal skin appearance using any dermatological textbook, for example, HISTOPATHOLOGY OF THE SKIN, Lever and Schaumburg-Lever (eds.), J.B. Lippincott Company (1991) and TEXTBOOK OF DERMATOLOGY, Champion et al. (eds.), 5th Ed. Blackwell Scientific Publications (1992), “Anatomy and Organization of Human Skin”; PHYSIOLOGY, BIOCHEMISTRY AND MOLECULAR BIOLOGY OF THE SKIN, VOLS. I AND II, Goldsmith (ed.), Oxford Press (1991), relevant portions incorporated herein by reference.

As used herein, the term “pharmaceutically-” or “therapeutically-acceptable” refers to a substance that does not interfere with the effectiveness or the biological activity of the active ingredients, which is not toxic to the host. The host to which it is administered may be either humans or animals.

As used herein, the term “therapeutically-effective amount” refers to the amount of an active agent sufficient to induce a desired biological result. That result may be alleviation of the signs, symptoms, or causes of a disease, or any other desired alteration of a biological system. As used in conjunction with the present invention, one method for determining the effectiveness is by evaluating one or more skin conditions in a patient selected from at least one of decreased pH, inflammation, comedon, acne, hyperkeratosis, enlargement of sebum vessels, pores enlargement, piling centers, skin discoloration or scars.

As used herein the term “nutritionally effective amount” is used to define the amount that will provide a beneficial nutritional effect or response in a mammal. For example, as nutritional response to anti-oxy-vitamin lotions, creams and/or dietary supplements varies from mammal to mammal, it should be understood that nutritionally effective amounts of the vitamins and minerals will vary, respectively.

As used herein, the term “acceptable salt” refers to those salts that are, within the scope of sound medical judgment, suitable for use in, on or with the tissues of humans and lower animals without undue toxicity, irritation, allergic response and are commensurate with a reasonable benefit/risk ratio. Acceptable salts are well-known in the art (see e.g., S. M. Berge, et al., J. Pharmaceutical Sciences, 1977, relevant portions incorporated herein by reference) and may be prepared during the final isolation and purification of the compounds of the invention or separately by reacting a free base function with a suitable organic acid. Representative acid addition salts include, but are not limited to acetate, adipate, alginate, citrate, aspartate, benzoate, benzene sulfonate, bisulfate, butyrate, camphorate, camphor sulfonate, digluconate, glycerophosphate, hemisulfate, heptanoate, hexanoate, fumarate, hydrochloride, hydrobromide, hydroiodide, 2-hydroxyethansulfonate (isothionate), lactate, maleate, methane sulfonate, nicotinate, 2-naphthalene sulfonate, oxalate, palmitoate, pectinate, persulfate, 3-phenylpropionate, picrate, pivalate, propionate, succinate, tartrate, thiocyanate, phosphate, glutamate, bicarbonate, p-toluene sulfonate and undecanoate. Examples of basic nitrogen-containing groups that are used as quaternizing agents include: lower alkyl halides (methyl, ethyl, propyl, and butyl chlorides, bromides and iodides); dialkyl sulfates (dimethyl, diethyl, dibutyl and diamyl sulfates); long-chain halides (decyl, lauryl, myristyl and stearyl chlorides, bromides and iodides); arylalkyl halides (benzyl and phenethyl bromides) and the like. Examples of acids that may be employed to form pharmaceutically acceptable acid addition salts include inorganic acids, e.g., hydrochloric acid, hydrobromic acid, sulphuric acid, and phosphoric acid and such organic acids as oxalic acid, maleic acid, succinic acid, and citric acid. Basic addition salts can also be prepared in situ during the final isolation and purification of anti-oxidant compounds disclosed herein with a suitable base such as the hydroxide, carbonate or bicarbonate of a pharmaceutically acceptable metal cation or with ammonia or an organic primary, secondary or tertiary amine. Pharmaceutically acceptable salts include, but are not limited to, cations based on alkali metals or alkaline earth metals such as lithium, sodium, potassium, calcium, magnesium and aluminum salts and the like, and nontoxic quaternary ammonia and amine cations including ammonium, tetramethylammonium, tetraethylammonium, methylammonium, dimethylammonium, trimethylammonium, triethylammonium, diethylammonium, and ethylammonium among others. Other representative organic amines useful for the formation of base addition salts include ethylenediamine, ethanolamine, diethanolamine, piperidine, piperazine, and the like.

Techniques and compositions for making useful dosage forms using the present invention are described in one or more of the following references: Ansel, Introduction to Pharmaceutical Dosage Forms 2nd Edition (1976); Remington's Pharmaceutical Sciences, 17th ed. (Mack Publishing Company, Easton, Pa., 1985); Advances in Pharmaceutical Sciences (David Ganderton, Trevor Jones, Eds., 1992); Advances in Pharmaceutical Sciences Vol 7. (David Ganderton, Trevor Jones, James McGinity, Eds., 1995); Aqueous Polymeric Coatings for Pharmaceutical Dosage Forms (Drugs and the Pharmaceutical Sciences, Series 36 (James McGinity, Ed., 1989); Pharmaceutical Particulate Carriers: Therapeutic Applications: Drugs and the Pharmaceutical Sciences, Vol 61 (Alain Rolland, Ed., 1993); Drug Delivery to the Gastrointestinal Tract (Ellis Horwood Books in the Biological Sciences. Series in Pharmaceutical Technology; J. G. Hardy, S. S. Davis, Clive G. Wilson, Eds.); Modern Pharmaceutics Drugs and the Pharmaceutical Sciences, Vol 40 (Gilbert S. Banker, Christopher T. Rhodes, Eds.), and the like, relevant portions incorporated herein by reference.

The present invention includes compositions and methods for preventing and treating skin conditions using a topical composition that may also include a pharmaceutically acceptable carrier. Carriers for topical application useful in practicing the invention include those well-known to those of skill in the art as well as those outlined in the examples disclosed herein.

As used herein, the term “pharmaceutically acceptable” refers to molecular entities and compositions that are not toxic and/or do not produce an allergic or similar untoward reaction when administered to a human. Pharmaceutically acceptable carriers and additives employed in the present compositions are compatible with at least one formulation of the anti-oxy-vitamins, including lactoferrin, conjugated linoleic acids, Hippohae rhamnoides L. berry extract, omega fatty acids, low molecular weight whey protein extract, vitamin E and antocyanes.

The lactoferrin, conjugated linoleic acids, Hippohae rhamnoides L. berry extract, omega fatty acids, low molecular weight whey protein extract, vitamin E and antocyanes, employed in the present compositions will be as pyrogen free as possible and should meet sterility, pyrogenicity, general safety and purity standards as required by FDA Office of Drug standards. The composition may also be buffered. Illustrative examples of the present compositions can be produced as follows. The appropriate amounts of the acid forms of native and untreated anti-oxy-vitamins are mixed and/or dissolved in water or lipid solvent. The resulting solution can be employed as a topical composition in the form of a cream, lotion, gel, paste or serum produced by any of a variety of conventional formulations well known to those skilled in the art and as disclosed herein.

The present topical composition may be in the form of an aqueous solution or blended into a tissue compatible vehicle, such as hydrophilic lotion-, ointment-, cream- or gel-based vehicle. Such vehicles are well known in the art and commercially available for formulation of active ingredients into a suitable form for topical application.

The present composition may also include one or more of a variety of optional ingredients, such as coloring agents, scents, opacifying agents and the like. The formulation may also include: other active ingredients, antibiotics, analgesics, anti-allergenics and the like. The formulation is commonly applied to the skin as a lotion or cream to be rubbed on body tissue over the desired area. For optimum efficacy treatment in accordance with the presented method should be initiated as early as possible following exposure to sunlight or another radiation source. The formulation is generally applied to the skin once or twice daily. As noted elsewhere herein, the present composition may also be used to inhibit the effects of aging and/or photo damage on the skin.

Upon formulation, the compositions of the present invention may be administered in a manner compatible with the dosage formulation and in such amount as is therapeutically effective. For topical administration in an aqueous solution, for example, the combination of lactoferrin and conjugated linoleic acids (and optionally one or more of the following: Hippohae rhamnoides L. berry extract, omega fatty acids, low molecular weight whey peptides extract, vitamin E and antocyanes), may be used directly on the skin without any toxic or allergic effects to the animal or patient. Alternatively, the medicinal progression of anti-oxy-vitamin compositions may be dissolved or resuspended in a suitable buffer prior to mixing, if necessary. Liquid diluents may first be rendered isotonic with sufficient saline or glucose solutions. The addition of one or more of the following: Hippohae rhamnoides L. berry extract, omega fatty acids, low molecular weight whey protein extract, vitamin E and antocyanes will depend on the level of free-radical damage to the skin (or to prevent the damage) as taught hereinbelow.

The present aqueous solutions are especially suitable for topical administration. As discussed above, however, other anti-oxy-vitamin-based formulations may also be used quite effectively. Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

The present invention also includes non-topical forms of the anti-oxy-vitamins that may be used in conjunction with the topical form to synergize with the topical formulation. Examples of dosage form for providing the anti-oxy-vitamins concomitantly with the topical formulations include: oral, intravenous (bolus or infusion), intraperitoneal, subcutaneous, or intramuscular form, all using dosage forms well known to those of ordinary skill in the pharmaceutical arts. Depending on the particular location or method of delivery, different dosage forms, e.g., tablets, capsules, pills, powders, granules, elixirs, tinctures, suspensions, syrups, and emulsions may be used to provide the anti-oxy-vitamins of the present invention to a patient in need of prevention, curing or treating the damage caused by free radicals. The anti-oxy-vitamins may also be administered as any one of known salt forms.

The skin is the largest organ in human and mammalian body. The principal functions of the skin include protection, excretion, secretion, absorption, thermoregulation, sensory perception and regulation of the immunological defenses (mechanisms) protecting a body from microbial invasion. These functions are all affected by the structural changes in the skin with aging.

The physiological changes associated with aging (these reductions) include impairment of the barrier function, decreased turnover of epidermal cells, reduced numbers of keratinocytes and fibroblasts, and a reduced vascular network. With regard to the “love” of outdoor activity of the contemporary population, modern sunscreens should provide high and broad-spectrum of the ultraviolet protection in the ultraviolet B as well as in the ultraviolet A range, and should be photochemically stable for ultraviolet doses, which can be expected in solar radiation. The ultraviolet light augments production of different kind of free radicals, in particular the one that is mainly responsible for the accelerated aging of the skin and body. Prevention of skin senescence relies in part on application of nutricosmetics, which would neutralize formation of the negative factors like free radicals in particular. One of the most important elements of the newly designed cosmetics called here Biovien® series is there capability to achieve this goal through control of free radicals, through adequate combinations of Vitamins E and C, A, (its precursors), as well as, a group of peptides present in body fluids (like milk) and some plant extracts showed positive effects in the skin protection.

The present invention is directed to a novel progressive administration of topical and oral (diet supplements) “cosmetics” to prevent and modulate dermatological conditions and metabolic processes or disorders in humans. More specifically, the present invention is directed to the simultaneous administration of combinations of antioxidant adjusted to the age of user in a topical formulations such as cream or body lotion (body milk) to the skin of humans and oral administration (diet supplements). Together the topical and oral administration of the same active principles improves cosmetics and metabolic performance. These improvements are called now the amplification phenomenon and represent a novel approach for overall health, not just the skin. It was found that the compositions and methods of the present invention provide a synergetic effect not only on the skin condition, but also act as an essential element needed for slowing aging. The progression of compositions of the present invention involve the addition of other biologically active compounds to bovine lactoferrin in the following manner: 1) lactoferrin, fatty acids; 2) lactoferrin, fatty acids, conjugated linoleic acids (CLA) and vitamin E; 3) lactoferrin, fatty acids, C.L.A and plant-derived lipid like Hippohae rhamnoides L. berry extract (“oblepicha”) and vitamin E; 4) lactoferrin, fatty acids, plant-derived lipid, “oblepicha”, omega fatty acid and vitamin E; 5) lactoferrin, CLA, plant-derived lipid, “oblepicha”, omega fatty acids, low molecular weight whey protein extract and vitamin E. 6) The same biologically active elements only in different proportion. 7) the same biologically active elements as in the 6 only new antioxidant was added—Antocyanes. Mentioned above antioxidant complexes when delivered topically on the skin and orally has amplification properties.

The present invention includes novel compositions and methods for making and using the compositions in a synergistic effect. It has been found that the simultaneous delivery of the same biologically active compound on the skin and internally (diet supplements) had a synergistic effect on study subjects. The present combinations were also developed as a progression of compositions that are altered as the metabolism of the subject changes, more particularly, as seen during aging. It was also found that the compositions, progression and methods of the present invention had a profound effect on scar production, acne, skin blemishes and lesions, overall skin health and wrinkles. These combinations were designed according to the needs of the whole body for active elements in the different stages of the life.

One embodiment of the present invention includes the addition of one or more active agents or ingredients. It was found that introduction of different classes of biologically active substances in one formulation of cream and capsules and body lotion; anti-bacterial, maintenance antioxidant and rejuvenating; clinical studies were conducted to compare and contrast the effects demonstrated herein. There are 200 different types of cells that combine together, form organism that consist of 10¹⁷ cells, which is a human organism.

The present invention includes agents that demonstrate an antioxidant effect not previously described. The antioxidant complexes control the level of free radicals. Among antioxidant complexes used in this patent are: antioxidants that control production of free radicals, a group specialized in neutralization already formed free radicals and finally, inhibiting production of free radicals inside the cells. By applying the combinations of these three classes of antioxidants a series of compositions were developed and products tested that are efficiently protecting the human body against damaging affects of free radicals.

It was found that the production of free radicals increases with age. As such, it was necessary to develop and provide the human skin with different and more potent combinations of the scavengers, which were introduced in a progression at different phase of the life of the subject and/or to address specific challenges to the skin, e.g., prolonged exposure to UV light and other agents or conditions that cause an increase in free-radical production. Non-limiting examples of agents that increase free-radical production include irritants (organic and inorganic agents), heat, cold, mechanical disruptions (scrapes, bruises), exposure to harsh climates, irradiation, chemical treatments, bacteria, viruses, fungi, auto-immune disorders, anti-inflammatory disorders and cancer.

Combination of Lactoferrin, fatty acids and the conjugated linoleic acids need to be consider as a new invention, in the field of anti-oxidant use. Lactoferrin as inhibitor of the iron ions oxidation process, permit to claim, that “hiding” iron ions inside-lactoferrin, inhibits production of free radicals. The oxidation process releases quantity of free energy utilized by bacterial flora, growing on the top of the skin. On top, the Lactoferrin serve as an antibiotic for certain germs (double action). The use of the conjugated linoleic acids permit this compound to act as a classical scavenger, which neutralize existing free radicals, helping fatty acids in its scavenger activity. Combining both elements, Lactoferrin with conjugated linoleic acids together resulted in more efficient elimination of free oxidants from the body exhibiting synergistic effect. This phenomenon is termed hereinafter as an “amplification phenomenon”. It was found that the addition of vitamin E further strengthen the control level of free radicals.

Application of method used in this invention that inhibits free radicals formation by limiting their production, simultaneously with neutralization of the already existing free radicals represents new invention.

The successive addition of fatty acids, to other scavengers like conjugated linoleic acids, anthocyans, polyphenoles, carotenoids, omega fatty acids, peptides and lactoferrin, forms the “pyramid strategy”. This application of the antioxidant formulas represent another invention, because it augments the antioxidant activity, protects the immune defenses, improve control of chronic disorders.

Simultaneous application of the specific antioxidants formula delivered through topical and orally (in form of dietary supplement), in ranges that are safe and not toxic for humans represent new invention. The formulated complexes referred to herein as Biovien® cream 1, 2, 3, 4, 5, 6, 7 with Biovien™ 1, 2, 3, 4, 5, 6 of this invention broaden the application to modulation of diverse conditions, according to needs, that occur on skin and the body at different period of life. The broadening the applications represent a new invention. Simultaneous application of those combinations to cosmetics (soft gel capsules) and topical preparations (creams & lotions), adds extraordinary value to prevention and modulation conditions caused by oxidative stress. Management of the oxidative stress by using the compositions and methods of the present invention increased the opportunity for modulation of diverse disorders by controlling the damaging action of free radicals at three different levels: inhibit the production of free radicals, neutralize existing free radicals, and control production of free radicals inside the cell, which contributes to preventing damages done by free radicals.

Application, of the same biologically active substances simultaneously on the skin in form of a cream or body lotion and orally in form of soft gel capsules (dietary supplements) represents new invention. It represents new strategy to control oxidative processes, constantly occurring on the skin and the body.

It was found that there are people, who use cream with only one group of anti-oxidant in it and different antioxidant in form of dietary supplements. Such strategy is not necessarily complementary to each other. Since different scavengers work well only in specific combinations the used in indiscriminate fashion is not scientifically sound proposition. Presented here method represents a new approach to control free radicals and is based on sound scientific information.

Focusing on the hydroxyl free radical group in this invention, as a main target for the formula of this patent was dictated by fact, that this group of hydroxyls represents the most dangerous and difficult elements to protect the organism from. By delivering orally the dietary supplement (in form of capsules) it was found that the present invention protects the whole human organism. These compositions provide an effective method in the battle with excess of free radicals.

To further evaluate the enhanced effects of the present invention on skin treatments with serum and cream, some subjects were also provided the same composition orally. Further, the skin requirement for nutrients changes with the age. In order to meet the demand at different life stages, seven different creams formulas, six kinds of capsules/dietary supplements and seven forms of body lotion has been designed.

Gradual enrichment of cosmetics directed for clients at different age, represent addition invention. Requirements are changing with age and require a broader spectrum of nutrients. These rules are perhaps recognized by cosmetologist, but not expressed clearly to and did not apply to the whole body. Using above mentioned compositions and methods, favorable conditions were created for successful control of the oxidative stress affecting the body conditions manifested by increased well being of patients, increased patient compliance with treatment, increased self-esteem and better overall health.

Inclusion of anti bacterial, nutrient and rejuvenation factors in different stages of the life is another new invention. The combination was made according to a new paradigm, with a clear understanding of what components of the cosmetics are essential at given stage of the life. For example, mixing lactoferrin with colostrum, as some people are proposing represents superficially good idea, however, colostrum already includes a very high amount of lactoferrin. Further addition of lactoferrin is unlikely to have any effect whatsoever as the lactoferrin present in colostrum will cover the effect of added lactoferrin, may only cause Tachophilaxis. It was also found that a return to natural or native products may help with the synergistic effect on the body's largest organ, the skin. It was also found that the compositions and methods of the present invention can be effective to lessen the effect of allergies and their recurrence. Only very few people realized, how serious problem, allergies are for whole body and skin. The importance, become even more acute from time when new nutrition principles were adapted for “rational children growth” and a very broad use of cosmetics.

Simultaneous applications of natural elements, known from ages as hypoallergenic and having in addition antioxidant properties were also developed. These observations were used to design a complete progression or series of cosmetics that not only repair but also prevent damage of the skin and slow down the aging processes of the body. It was found that the cosmetics and dietary supplements disclosed herein act simultaneously as antioxidant, anti bacterial, anti wrinkle agent for the total body including skin.

Compositions.

The present invention is directed to a novel topical and oral administration of diverse biologically active ingredients to prevent and treat dermatological conditions or disorders in humans. More specifically, the present invention is directed to the administration of lactoferrin in a topical material such as an ointment or cream to the skin of humans and also including an oral administration; together the topical and oral administration has a synergetic effect in the treatment of the skin. The progression of biologically active substances of the present invention are: 1) lactoferrin; 2) lactoferrin and a plant or animal derived lipid; 3) lactoferrin, a plant or animal derived lipid and Hippohae rhamnoides L. (“Oblepicha”); 4) lactoferrin, a plant or animal derived lipid, “Oblepicha”, and omega fatty acid; 5) lactoferrin, a plant or animal derived lipid, “Oblepicha”, omega fatty acid and low molecular weight whey peptides. 6) lactoferrin, CLA. Plant derived lipids, “Oblepicha”, omega fatty acid, low molecular weight whey peptides, antocyanes, flax oil, and vitamin E.

Biologically active compounds and sample formulations. Products for the present invention are made by starting with one active compound alone and progressing with additional specific biologically active ingredients, which provide specific effect to the prevention or alleviation of common conditions of human skin and body. These ingredients are biologically active substances, which form compounds that are administered in this invention in the form of cream, body lotion or internally in the form of soft gel capsules/dietary supplements.

The first biologically active compound used was lactoferrin; well documented protein recovered from human, bovine, sheep, or porcine milk or colostrums. A preferred lactoferrin is that recovered from bovine milk.

The second selected products are fatty acids. They were provided from different commercially available oil, preferably at least one of Sunflower oil, Soy bean oil or Grape seeds oil.

The third selected product used-was conjugated linoleic acids (CLA). CLA has the structure: cis-9, trans-11 (C18: 2), with the double bonds separated by only one single bond. The preferred CLA is a natural product obtained from sunflower seeds.

The fourth selected product is a plant-derived lipid extract, from the fruits of Hippohae rhamnoides L. called also “oblepicha”.

The fifth selected product includes omega fatty acids that may be provided as an oil obtained from, e.g., flax seeds.

The sixth selected product is vitamin E.

The seven selected product are antocyanes. They are present in many fruits and colored berries, preferred in this invention: from aronia fruits. Antocyanes belong to a group of very strong scavengers of free radicals.

Finally, the last one derives from whey, and represents a low molecular weight fraction from about 1 to 15 kilodaltons, from 2 to 14 kilodaltons or from 3 to 14 kilodaltons. The present invention is directed to the progression of use of these ingredients to show that each has a specific function and that favorable results are obtained when the specific ingredients are used in a specific formulation for specific skin conditions at specific period of the life.

To illustrate the present invention, all the above biologically active substances (Lactoferrin, CLA derive from sunflower seeds, Hippohae rhamnoides L, Fatty acids, oil obtained from Flax seeds, called omega fatty acids, Vitamin E, Antocyanes, Proline Pich Peptides complex) are forming a proprietary blend that is part of the antioxidant complexes that are part of this invention, incorporated in Biovien® topical and oral formulas—Biovien™ dietary supplements.

Biovien®1-cream for young acne prone skin. The Biological Active Substance (BAS), in the Biovien®-1 cream, act as an inhibitor of skin germs. Lack of free iron is the reason why Biovien® products always inhibit iron dependent bacterial growth and prevent skin infections and blemishes. The antioxidant present in Biovien®-1 cream, controls the level of free radicals. Simultaneous application of Biovien®-1 cream, Body lotion and Biovien®-1 dietary supplements are suggested to prevent and control the skin infections and shorten significantly the time of effects appearance. Application: apply the cream on washed skin every day. Repeat procedure 2-times daily. Use the cream on existing blemishes until all disappear. Use as prevention.

Ingredients/International Nomenclature Cosmetic Ingredient (INCI):

### Aqua, up to 100%.

### PEG-35 Castor oil, 0.1-10%

### Glycerin, 0.1-1q0%

### Ethylhexyl stearate, 0.1-10%

### Polyacrylamide C-13-14 Isoparaffin, 0.1-10%

### Laureth-7, 0.1-10%

### Propylene glycol, 0.1-10%

### Centurea cyanus, 0.1-10%

### Vitis vinifera 1-10%

### Lactoferrin, 0.1-10%

### MDMD Hydantoin, 0.1-10%

### 3-Iodo Propynylo Carbamate, 0.1-10%

### Dimethicone. 0.1-10%

BIOVIEN®-2 CREAM for advanced stages of the bacterial skin infections (ACNE), the BAS in Biovien®-2 cream, in conjunction with a second BAS, third and forth BAS (FATTY ACIDS, CLA, VIT.E., LACTOFERRIN.) activates the humoral and the cellular arms of the defenses. The combination successfully inhibits germ growth-by making inaccessible iron ions and inhibiting its utilization by germ. In addition, Biovien®-2, due to the significant antioxidant power might be used for healthy, young skin also as a preventive factor. The combinations of the Biovien®-2 cream, the body lotion and the dietary supplements correct the metabolism. Simultaneous application of above combination, significantly shortens the time between the beginning of the application of Biovien® product and appearance of improvement. Application: Apply the cream on washed skin every day. Repeat procedure 2-times a day. Use the cream on existing blemishes until all disappear.

Ingredients/INCI:

### Aqua, up to 100%.

### Glycerin, 0.1-10%

### PEG-35 Castor oil, 0.1-10%

### Polyacrylamide, C13-14 Isoparaffin, 0.1-10%

### Laureth-7, 0.1-10%

### Cetearyl alcohol, 0.1-10%

### Soya bean oil, 0.1-10%

### Acetylated Lanolin, 0.1-10%

### Ethylhexyl stearate, 0.1-10%

### Paraffinum Liquidum, 0.1-10%

### Vitis Venifera Oil, 0.1-10%

### Lactoferrin, 0.1-10%

### C.L.A., 0.1-10%

### Tocopheryl acetate. 0.1-10%

### DMDM Hydantoin, 0.1-10%

### 3-Iodo Propynylo Bytulo Carbamate, 0.1-10%

### Dimethicone, 0.1-10%

BIOVIEN®-3-ANTIWRINKLE CREAM, PROTECTS AGAINST HARMFUL ENVIRONMENTAL DAMAGE. Biovien™-3 is recommended for young and adult people that love outside activities. In addition to its ability to inhibit bacteria growth, Biovien®-3 has an increased amount of biologically active substances (BAS)— that augmented the antioxidant complex, creating a potent scavenger complex. A rejuvenating agent protects the body and skin from a harmful environment. Combination Biovien®-3 cream, body lotion and dietary supplements slow the aging processes; providing the body and skin with vitamins, anti-oxy vitamins, precursors of vitamin A, and the transition metals. The above combination, permits the formation of a thin layer of lipid membrane, that prevents the dehydration of the skin and protects against ultraviolet damages. Simultaneous use of Biovien®-3 cream, body lotion and dietary supplements has potentiating effect. Application: Apply the cream on skin every day. Repeat procedure 2-3 times daily.

Ingredients/INCI:

### Aqua, up to 100%.

### Citric acid, 0.1-10%

### Sodium citrate, 0.1-10%

### Glycerin, 0.1-10%0.1-10%

### Centenary Alcohol, 0.1-10%

### Soybean oil, 0.1-10%

### Ethylhexyl Stearate, 0.1-10%

### Vitis vinifera oil, 0.1-10%

### Hippohae rhamnoides L. 0.1-10%

### Lactoferrin, 0.1-10%

### C.L.A., 0.1-10%

### Tocopheryl acetate. 0.1-10%

### Ceatereth-25, 0.1-10%

### Ceteareth-6, 0.1-10%

### Triethanoloamine, 0.1-10%

### DMDM Hydantoin, 0.1-10%

### 3-Iodo Propynylo Butylo Carbamate, 0.1-10%

### Carbomer, 0.1-10%

### Dimethicone, 0.1-10%

BIOVIEN®-4 REGENERATING FORMULA-DEFENDS AGAINST THE AGING PROCESS: Designed for dry, sensitive adult skin; recommended particularly for regenerating and an anti-aging shield. Along with its antibacterial properties and enhanced antioxidant power the Biovien®-4 cream has new elements particularly important for augmentation of the regeneration processes and control of free radicals. The present combination (with Biovien™-4, dietary supplements and body lotion) provides the host with the elements most frequently absent in today's diet. The addition of a new active substance (BAS) to Biovien®-4 products resulted in a stronger anti-aging defense. Simultaneous application of the Biovien®-4 cream, body lotion and dietary supplements, significantly shortens the time between application of Biovien® cream and appearance of the effects. Application: apply cream on washed skin every day. Repeat procedure 2-times daily.

Ingredients/INCI:

### Aqua, up to 100%.

### Citric acid, 0.1-10%

### Sodium citrate, 0.1-10%

### Glycerin, 0.1-10%

### Lactoferrin, 0.1-10%

### Soybean oil, 0.1-10%

### Vitis vinifera oil, 0.1-10%

### Hippohae rhamnoides L., 0.1-10%

### Flax oil, 0.1-10%

### Tocopheryl acetate. 0.1-10%

### Ethylhexyl stearate, 0.1-10%

### Ceteareth-25, 0.1-10%

### Ceteareth-6, 0.1-10%

### Cetearyl Alkohol, 0.1-10%

### DMDM Hydantoin, 0.1-10%

### 3-Iodo Propynylo Butylo Carbamate, 0.1-10%

### Dimethicone 0.1-10%

BIOVIEN®-5 RICH ANTI-AGING CREAM-ANTIOXIDANT BOOSTER. Recommended for seriously damaged skin exposed to unfriendly environment, stressful conditions, and physico-chemical traumatized skin or chronic infections. In addition to antioxidants, anti bacterial, and rejuvenating substances, the Biovien™-5 is enriched with a complex of low molecular weight peptides. The peptides also augment the control the free radicals. The neutralization of free radicals is the key to rejuvenation the skin. Production of free radical's increases with age, therefore, more potent combination of the scavengers is recommended for adults to regain control of the aging and regenerating processes. The simultaneous use of the Biovien®-5 cream, body lotion and dietary supplements (capsules) are designed to correct changes occurring in adult body and skin. Applying our new philosophy of simultaneous application gives the potentiating effect.

Application: apply the cream on washed skin every day. Repeat procedure 2 times daily.

Ingredients/INCI:

### Aqua, up to 100%.

### Citric acid, 0.1-10%

### Sodium citrate, 0.1-10%

### Glycerin, 0.1-10%

### Lactoferrin, 0.1-10%

### Soybean oil, 0.1-10%

### Hippohae rhamnoides L., 0.1-10%

### Vitis venifera oil, 0.1-10%

### Flax oil, 0.1-10%

### Whey protein, 0.0001-10%

### Tocopheryl acetate 0.1-10%

### Ethylhexyl stearate, 0.1-10%

### Ceteareth-25, 0.1-10%

### Ceteareth-6, 0.1-10%

### Cetearyl Alkohol, 0.1-10%

### DMDM Hydantoin, 0.1-10%

### 3-Iodo Propynylo Butylo Carbamate, 0.1-10%

### Dimethicone 0.1-10%

BIOVIEN®-6 REJUVENATING CREAM, RICH IN ACTIVE INGREDIENTS AND AN EFFECTIVE REPAIR COMPOUND. Recommended for mature skin damaged by environmental conditions, distressed by medical treatment, and/or excessive damages due to age, cosmetic procedures or chronic inflammations. Composition of the BAS in Biovien®-6 products is identical to Biovien®-5; only the percentile proportion was changed in favor of the Biovien®-6. Production of free radical's increases with age in a stressful environment, therefore a more powerful combination of scavengers is needed. Changes occurring in adult body and skin as a result of excessive damages due to age, environmental damages, or distressed by medical conditions (high energy radiation), cosmetic procedures and chronic infections, will be helped by use of Biovien®. The combinations of the Biovien®-6 cream, the body lotion and the dietary supplements is formulated to help regain the optimum health of the skin. It is achieved by providing the required factors needed to stimulate the natural repair and rejuvenating mechanism. These conditions are frequently accompanied with severe pain. Biovien®-6 cream is effective in reducing or eliminating pain of the damage skin. Simultaneous application of Biovien® cream, body lotion and capsules shortens the time needed for improvement. Application: Apply the cream on washed skin every day. Repeat procedure 2-3 times daily.

Ingredients/INCI:

### Aqua, up to 100%.

### Citric acid, 0.1-10%

### Sodium citrate, 0.1-10%

### Glycerin, 0.1-10%

### Soybean oil, 0.1-10%

### Lactoferrin, 0.1-10%

### Soybean oil, 0.1-10%

### Hippohae rhamnoides L., 0.1-10%

### Vitis venifera oil, 0.1-10%

### Flax oil, 0.1-10%

### Whey protein, 0.0001-10%

### Tocopheryl acetate 0.1-10%

### Ethylhexyl stearate, 0.1-10%

### Ceteareth-25, 0.1-10%

### Ceteareth-6, 0.1-10%

### Cetearyl Alkohol. 0.1-10%

### DMDM Hydantoin, 0.1-10%

### 3-Iodo Propynylo Butylo Carbamate, 0.1-10%

### Dimethicone, 0.1-10%

Biovien® Mask. For application 1 time per weak.

Ingredients/INCI:

### Aqua, up to 100%.

### Citric acid, 0.1-10%

### Sodium citrate, 0.1-10%

### Glycerin, 0.1-10%

### Lactoferrin 0.1-10%

### Soybean oil, 0.1-10%

### Vitis venifera oil, 0.1-10%

### Hippohae rhamnoides L., 0.1-10%

### Flax oil, 0.1-10%

### Whey protein, 0.0001-10%

### Tocopheryl acetate. 0.1-10%

### Antocyanes 0.01-1.0%

### Ethylhexyl stearate, 0.1-10%

### Ceteareth-25, 0.1-10%

### Ceteareth-6, 0.1-10%

### Cetearyl Alkohol. 0.1-10%

### DMDM Hydantoin, 0.1-10%

### 3-Iodo Propynylo Butylo Carbamate, 0.1-10%

### Dimethicone, 0.1-10%

Dietary Supplement/Capsules:1-6

BIOVIEN™-1 Dietary supplements. For young body and skin. Prevents or Controls bacterial infections, stimulates the immune system. Control production of free radicals, amplified the Biovien® creams and Body Lotion action. Take one capsules ones or twice daily with meal. Maximal effect will be seen after three cycles (90 days of use).

Ingredients INCI:

### Vitis vinifera oil, 0.1-50%

bovine lactoferrin, 0.1-10%
p-hydroksybenzoesan, 10-20%
amorfic silica oxide, 1-50%
fish gelatin, to make 0.5 g capsule.

Biovien™-2 Dietary supplements. Prevents or Controls severe bacterial infections—Acne, by stimulating cellular and humoral immune processes. The formula is designed to prevent or control existing severe acne infection and to control production and level of the free radicals. Due to significant antioxidant power can be used as preventing factor-for healthy skin. Supports Biovien®-2 cream. Take: one capsules ones or twice a day, with meal. Maximal effect will be seen after three cycles (90 days of use).

Ingredients INCI:

p-hydroksybenzoesan, 10-20%
lactoferrin, 0.1-10%

### C.L.A., 0.1-10%

### Vitis vinifera oil, 0.1-50%

### Tocoferol acetate, 0.1-10%

amorfic silica oxide, 1.0-50%
fish gelatin, to make 0.5 g capsule.

Biovien™-3 Dietary supplements. Rich in vitamin, pro-vitamins A, anty-oxy-vitamins, antioxidants. The formula is designed for athletic adults. Protects from harmful environment, rich in anti-microbial factors, provides a powerful antioxidant complex—controls chronic processes and maintains optimal health of the whole body and skin. Take: one capsules ones or twice a day, with meal. Maximal effect will be seen after three cycles (90 days of use).

Ingredients INCI:

### Vitis vinifera oil, 0.1-50%

### Hippohae Rhamnoides L., 0.1-10%

### Bovine lactoferrin, 0.1-10%

### C.L.A, 0.1-10%

### Tocoferol acetate, 0.1-10%

p-hydroksybenzoesan, 10-20%

### Amorfic silica oxide, 1.0-50%

### Fish gelatin. to make 0.5 g capsule.

Biovien™-4 Dietary supplements. Provides strong anti-oxidants complex and anti-inflammatory property, particularly useful for controlling chronic, systemic, and infectious disorders. Recommended for dry sensitive skin, damaged by incorrect management. The Anti oxidant complex neutralizes and inhibits free radicals formation due to the presence of metallo-proteins, anti-oxy-vitamins and vitamins. Take: one capsules ones or twice a day, with meal. Maximal effect will be seen after three cycles (90 days of use).

Ingredients INCI:

### Vitis vinifera oil, 0.1-50%

### Hippohae Rhamnoides L., 0.1-10%

### Bovine Lactoferrin, 0.1-10%

### Flax oil, 0.1-10%

### C.L.A, 0.1-10%

### Tocoferol acetate, 0.1-10%

### Amorfic silica oxide, 1.0-50%

p-hydroksybenzoesan, 10-20%

### Fish gelatin to make 0.5 g capsule.

BIOVIEN™ 5 Dietary supplements. Represents powerful complex of anti-oxidants controlling free radicals generated by the body and skin. Strong regenerating complex regulating cellular metabolism and controls inflammatory processes by activating native and immune defense processes. Take: one capsules ones or twice a day, with meal. Maximal effect will be seen after three cycles (90 DAYS OF USE).

Ingredient INCI:

### Vitis vinifera oil, 0.1-20%

### Bovine Lactoferrin, 0.1-10%

### Hippohae Rhamnoides L., 01-10%

### Flax oil, 0.1-10%

### Milk peptides, 0.0001-10%

### C.L.A, 0.1-10%

### Tocoferol acetate, 0.1-10%

### Amorfic silica oxide, 1.0-50%

p-hydroksybenzoesan, 1.0-20%

### Fish gelatin. to make 0.5 g capsule.

Biovien™ 6 Dietary supplements.

Ingredient INCI:

### Vitis vinifera oil, 0.1-50%

### Antocyanes 0.001-2%

### Hippohae Rhamnoides L., 01-10%

### Bovine Lactoferrin, 0.1-10%

### Flax oil, 0.1-10%

### Milk peptides, 0.0001-10%

### C.L.A. 0.1-10%

### Tocoferol acetate, 0.1-10%

p-hydroksybenzoesan, 1.0-20%

### Amorfic silica oxide, 1.0-50%

### Fish gelatin. to make 0.5 g capsule

Biovien® body lotions 1-6. 18.04.05.

Biovien®-1, Body lotion formulated for young skin to control bacterial infections and the prevention of blemishes. The Biologically Active Compounds (BAC), in the Biovien®-1 body lotion act as an inhibitor of the skin germs. Lack of free iron explains the reason, why Biovien®-1 body lotion inhibits bacterial growth. In addition it acts as an antioxidant controlling the level of free radicals. Apply the body lotion on washed skin, distribute uniformly on the surface. Repeat procedure twice a day. Use the body lotion until all blemishes and inflammations disappear.

Ingredients INCA:

### Aqua, up to 100%.

### Citric acid, 0.1-10%

### Sodium citrate, 0.1-10%

### PEG-35 Castor oil, 0.1-10%

### Vitis vinifera oil, 0.1-10%

### Lactoferrin, 0.10-10%

### Glycerin, 0.1-10%

### Ethylhexyl stearate, 0.1-10%

### Polyacrilamide, 0.1-10%

### C 13-14 Isoparaffin Laureth-7, 0.1-10%

### Propylene glycol, 0.1-10%

### Centurea cyanus, 0.1-10%

### Thriethanoloamine, 0.1-10%

### DMDM Hydantoin, 0.1-10%

### 3-Iodo Propenylo Butylo Carbamate, 0.1-10%

### Carbomer, 0.1-10%

### Dimeticone. 0.1-10%

Biovien®-2, Body Lotion formulated to effectively battle Acne. The body lotion successfully inhibits germ growth by making inaccessible, iron ions and inhibiting its utilization by germ. Lactoferrin in conjunction with CLA, activates the humoral and the cellular arms of the innate defenses. The BAS in Biovien®-2 body lotion controls the gland secretion by correcting the metabolism. In addition, due to the significant antioxidant power, Biovien®-2 body lotion might be used as a preventive formula for young skin. Apply the body lotion on washed skin, distribute uniformly on the surface. Repeat procedure twice a day. Use the body lotion until all blemishes and inflammations disappear.

Ingredients INCI:

### Aqua, as much as is needed.

### Citric acid, 0.1-10%

### Sodium Citrate, 0.1-10%

### Glycerin, 0.1-10%

### Soybean oil, 0.1-10%

### Vitis vinifera oil, 0.1-10%

### Lactoferrin, 0.1-10%

### C.L.A. 0.1-10%

### Tocoferyl acetate, 0.1-10%

### PEG-35 Castor oil, 0.1-10%

### Polyacrylamide C13-14 Isoparafin, 0.1-10%

### Laureth-7, 0.1-10%

### Cetearyl Alcohol, 0.1-10%

### Acetylated Lanolin, 0.1-10%

### Ethylhexyl stearate, 0.1-10%

### Parafinum liquidum, 0.1-10%

### MDMD Hydantoin, 0.1-10%

3-iodo Propynylo Butylo Carbamate, 0.1-10%

### Dimethicone. 0.1-10%

Biovien®-3 Body Lotion is formulated as anti-wrinkle—potent anti-oxidants. In addition to its ability to inhibit bacteria growth, body lotion acts as a potent scavenger complex. Also acts as a rejuvenating agent, slowing the aging processes and protecting body and skin from a harmful environment. The body lotion is helping the formation of a thin layer of lipid membrane that prevents the dehydration of the skin and protects against ultraviolet damages. Apply the body lotion on washed skin and distribute uniformly on the surface. Repeat procedure 2 times daily

Ingredients INCI:

### Aqua, up to 100%.

### Citric acid, 0.1-10%

### Sodium citrate, 0.1-10%

### Ethyhexyl stearate, 0.1-10%

### Glycerin, 0.1-10%

### Paraffinum perliquidum, 0.1-10%

### Ceteareth-25, 0.1-10%

### Ceteareth-6, 0.1-10%

### Cetearyl alcohol, 0.1-10%

### Dimethicone, 0.1-10%

### Vitis venifera oil, 0.1-10%

### Soybean oil, 0.1-10%

### Lactoferrin, 0.1-10%

### Hippohae rhamnoides L. oil, 0.1-10%

### C.L.A., 0.1-10%

### Tocoferyl acetate, 0.1-10%

### Carbomer, 0.1-10%

### Triethanolamine, 0.1-10%

### DMDM Hydantoin, 0.1-10%

### 3-Iodo Propanylo Butylo Carbamate, 0.1-10%

### Perfume. 0.1-10%

Biovien®-4, body lotion—regenerating formula designed for dry, sensitive adult skin. Recommended particularly as a regenerating power and an anti-aging shield. With its antibacterial properties and enhanced antioxidant power body lotion has new elements that are important for the regeneration processes. The present combination provides the host with the elements most frequently absent in today diet used by the mature population. Simultaneous application Biovien®-4 products; body lotion cream and dietary supplement, shorten significantly the time between the beginning and appearance the effect. Apply on washed skin the body lotion and distribute uniformly on the surface. Repeat procedure 2 times a day.

Ingredients INCI:

### Aqua, up to 100%.

### Citric acid, 0.1-10%

### Sodium citrate, 0.1-10%

### Ethylhexyl stearate, 0.1-10%

### Glycerin, 0.1-10%

### Paraffinum liquidum, 0.1-10%

### Ceteareth-25, 0.1-10%

### Ceteareth-6, Cetearyl alcohol, 0.1-10%

### Vitis vinifera oil, 0.1-10%

### Sunflower oil, 0.1-10%

### Hyppohae rhamnoides L. oil, 0.1-10%

### Flax oil, 0.1-10%

### Lactoferrin, 0.1-10%

### C.L.A., 0.1-10%

### Tocoferyl acetate, 0.1-10%

### Dimethicone, 0.1-10%

### Carbomer, 0.1-10%

### DMDM Hydantoin, 0.1-10%

### 3-Iodo Propynylo Butylo Carbamate, 0.1-10%

perfume. 0.1-10%

Biovien®-5, Body Lotion—Rich Anti-Aging formula: Anti-oxidant booster. Designed to correct changes occurring in the adult skin. Is recommended for damaged skin, exposed to unfriendly environment, stressful conditions, and physico-chemical agents or chronic inflammations. The Biovien™-5 body lotion is enriched with a complex of peptides. The peptides are needed as an information network for effective functioning of the skin. The peptides also augment the anti free radical power of the body lotion. More potent combination of the scavengers are recommended for adults to regain control over the aging and degeneration processes. The neutralization of free radicals is the key step for rejuvenation of the skin. Biovien® body lotion significantly shorten, the time needed for the effect appearance. Apply body lotion on washed skin, and distribute uniformly on the surface. Repeat procedure 2 times daily.

Ingredients INCI:

### Aqua, up to 100%.

### Citric acid, 0.1-10% Sodium citrate,

### Ethylhexyl stearate, 0.1-10%

### Glycerin, 0.1-10%

### Paraffinum liquidum, 0.1-10%

### Ceteareth-25, 0.1-10%

### Ceteareth-6, 0.1-10%

### Cetearyl alcohol, 0.1-10%

### Vitis vinifera oil, 0.1-10%

### Sunflower oil, 0.1-10%

### Hyppohae rhamnoides L. oil, 0.1-10%

### Flax oil, 0.1-10%

### Lactoferrin, 0.1-10%

### C.L.A., 0.1-10%

### Tocoferyl acetate, 0.1-10%

### Whey peptides, 0.0001-10%

### Dimethicone, 0.1-10%

### Carbomer, 0.1-10%

### DMDM Hydantoin, 0.1-10%

### 3-Iodo Propynylo Butylo Carbamate, 0.1-10%

perfume. 0.1-10%

Biovien®-6, Body Lotion—Rejuvenating, soothing sensitive skin; Simultaneously stimulates the body natural repair ability. Biovien®-body lotion is formulated for adult skin with excessive damages due to age, environmental conditions, distressed by medical condition, cosmetic procedures or chronic inflammation. Such conditions are frequently accompanied with severe pain. Biovien®-6 body lotion is fast and effective in elimination the pain. Composition of the BAS in Biovien™-6 body lotion is identical to Biovien™-5; only the percentile proportion was changed in favor of the Biovien™-6. Oxidative processes are seldom limited to the skin region, therefore the use of Biovien® body lotion is also highly recommended as a supporting application. The simultaneous application, the Biovien® body lotion, cream and dietary supplement shorten significantly a lag period between application and effect of the body lotion. Apply on washed skin, place the body lotion and distribute uniformly on the surface. Repeat procedure 2 times a day.

Ingredients INCI:

### Aqua, up to 100%.

### Citric acid, 0.1-10%

### Sodium citrate, 0.1-10%

### Ethylhexyl stearate, 0.1-10%

### Glycerin, 0.1-10%

### Dimethicone, 0.1-10%

### Ceteareth-25, 0.1-10%

### Ceteareth-6, 0.1-10%

### Cetearyl alcohol, 0.1-10%

### Vitis vinifera oil, 0.1-10%

### Hyppohae rhamnoides L. oil, 0.1-10%

### Flax oil, 0.1-10%

### Lactoferrin, 0.1-10%

### C.L.A., 0.1-10%

### Sunflower oil, 0.1-5%

### Whey peptides, 0.0001-10%

### Tocoferyl acetate, 0.1-10%

### Paraffinum liquidum, 0.1-10%

### Carbomer, 0.1-10%

### DMDM Hydantoin, 0.1-10%

### 3-Iodo Propynylo Butylo Carbamate, 0.1-10%

### Perfume 0.1-10%

### EXAMPLE-1

The aim of this study was to develop a line of nutri-cosmetics adapted to specific needs of the body and skin at different age. Among recognized elements affecting the appearance of the body and the skin are: maturation processes, dys-hormonosis, aging, inadequate nutrition, infections, degenerative processes and various assaults like wrong diet or stress. Most of the mentioned variables are related to an induced imbalance of free radicals in the body. Single or combinations of those variables requires preparation of different types of nutri-cosmetics covering the needs of an individual. Needed bioactive elements have to be simultaneously delivered to the skin and internally during the entire period of the life. The addition of bioactive elements in the form of cream, lotion and dietary supplements can fulfill the requirements for biologically active elements. The effect of the compositions and methods taught herein were tested to determine the effect of the active substances simultaneously for the nourishment and health of the body and the skin. In addition, it recognized the need to control the oxido-reduction reactions. Since production of free radicals begins from the birth of a human and intensifies with the age, the control of these damaging elements is necessary. Among the many skin problems affecting the human population, the oily skin, particularly when it is infected with a germ, represents significant importance among young individuals.

When designing the Biovien® products, G.C performed pre selective screening of the majority of biologically active candidates for the anti-oxidant complexes. The designed complexes for different age groups were further assessed on the volunteers. Generated information concerning safety was collected. In the primary pilot study, 69 volunteers were used to answer whether G.C. products are inducing adverse reactions, whether activating existing allergies, or inducing new allergic reaction.

The volunteers were selected in such a manner that a subgroup was sensitive to different biologically active substances or antigens. After three weeks of testing two products, B-1 and B-5, (the simplest and the richest), the volunteers assessed their values. All volunteers indicated an absence of any adverse reaction to the creams and no activation of allergic reaction or induction of new allergies.

The Biovien® product was designed to control the level of free radicals throughout the human life spun. The below listed definition of the free radical served as a guide in preparation of complexes controlling free radicals. The free radicals are ions or molecules that at their external orbit have unpaired electron.

In different situations, during the life span of a subject different internal and external agents lead to the initiation of excessive production of the free radicals. Unfortunately, the human body is rather unprepared to defend host against free radicals. The classical inducers of free radicals are germs. During their invasion, large quantities of free radicals are released, since invading microorganisms alters the host metabolism in order to provide supply of energy for their own purposes. Different classes of free radicals may appear and as a result of their negative impact; acute phases of infection can turn in to chronic form and initiate degenerative or inflammatory processes that shorten the life spun of the affected individual.

Today, there is no single product that could effectively protect an individual against the free radicals released during the life spun. Existing antioxidant products may be considered as relicts of the old believers that assumed one anti-oxidant can control every type of free radical. For that reason, claims concerning the existing antioxidant values of products on the market have to be considered as over assumptive. Consumption of different types of food, rich with a variety of antioxidants, can protect an individual against damages done by the free radicals. However, such strategy, although rational in practice is difficult to realize.

A series of scavenger complexes (anti oxidants) were developed that effectively cover a human body's needs during different situations occurring during the life span. When selecting the anti free radicals compound concentrated on those that will: control production of free radicals; neutralize free radicals already present in the body; and modulate intracellular metabolism. The best were selected from the three groups of anti oxidants and were used for the development of complexes. The variables listed below were used as criteria for the evaluation of the candidate. The scavenger properties were assayed using the Spin Trap Magnetic Resonance, the TEAC (Tralox method) and Fluorescein assay using 2′7′-dichloro dihydro fluorescein. Although, selected method did not provide identical results, they did provide useful information.

After passing the initial steps, candidates were further evaluated by the following methods: 1. Cyto-toxicity assay performed in concentrations of candidate from 0 to 500 μg/ml. 2. Test for measuring change in cell morphology. The assessment was carried out in cell grooving in vitro at concentration of tested sample identical to used in test 1. 3. Cell viability assay (Trypan blue excretion assay) was used on the cells grown in tissue cultures. Assay was performed at identical concentration as above. 4. Population doubling level (PDL) was carried at concentration of compound indicated above. 5. Effect on cell cycle progression was measured at concentration level indicated above. 6. Anti oxidant status was measured at different concentrations of the compound using fluorescein assay. 7. Effect on process of the apoptotic cell death induced by tested compound was also evaluated. 8. The Radioactive Thymidine incorporation was performed in order to learn how tested compound effected cell metabolism.

Results of the evaluation assays of the biologically active candidates lead to the rejection or selection of the candidate. Only those compounds that passed screening evaluation were used in future work on construction of the antioxidant complexes. The potency of the whole complex is dependent upon the combination of the individual antioxidants. To cover the individual needs for entire life spun: six complexes were constructed and called. Biovien® 1-6 (B®-1 to B®-6). They were constructed using reverse pyramid strategy, meaning that B®-1 was the simplest anti acne, and B®-6 the most complex and powerful-regenerating. Since the free radicals can be formed inside the body and on the skin's surface; three classes of products, having the same active components were designed; the creams, the body lotions, and the dietary supplements. During the pilot studies, only the creams were selected for evaluation purposes. The study permits us to answer, with a high level of confidence, whether G.C. products are toxic or affect the allergy. Before initiation of the clinical evaluation, selected products passed additional tests for microbiological contamination and mutagenicity. This last two were performed in the Institute of Food Safety Warszawa-Poland. Before the initiation of volunteer studies, Biovien® series were registered at the Institute of Medicine. (Noffer Institute) Registration provided permission to sell products in Poland and in the European Common Market.

For the Biovien® evaluation, a group of 69 volunteers recruited by the School of Cosmetology Medical University in Lodz, were selected. The studies presented in parts 1, 2, 3, and 4 provide answers to the following questions: 1. Whether designed product is toxic or affects allergic process. 2. Whether Biovien® 1-6 complexes are biologically active, safe and do not induce allergic reactions. 3. Whether application of the complexes on the skin and oral application of the Biovien™ dietary supplement, represent a more effective method to control free radicals; and 4. To find an objective method confirming values of the complexes. Generate objective proof of the product value. One such method was detecting changes in the skin pH.

In first study we provide the answer to the question concerning safety of the products and demonstrate that Bioivien® products do not induce allergy or activate dormant allergy. The rational for selection of the Biovien®-1 anti acne cream and the Biovien®-5 cream was to see whether the single biologically active substance present in B®-1 cream or the more rich antioxidant formula in Biovien®-5 induce adverse reactions. It is necessary to stress that we did not anticipate appearance of adverse reactions, since the applied compounds were natural and used for centuries by humans and were considered as safe.

Materials and Methods. The 69 volunteers recruited for evaluation purposes used the indicated Biovien® product for three months. Within this period volunteers had to observe whether tested products were toxic for the skin, or whether activated existing allergy or induced new allergic reactions. The Biovien® creams and body lotions were made by the AVA Laboratories in Warszawa Poland. The same routine elements needed for cream or body lotion production were provided by Ava Laboratories or Iraferon Co., Gdansk Poland. Studies were conducted at the Medical University School of Cosmetology in Lodz Poland.

Results:

TABLE 1
The age distribution in different volunteer's subgroups testing Biovien ® products:

| Group: | No | 14–20 | 21–25 | 26–30 | 31–35 | 36–40 | 41–45 | 46–50 | 51–55 | 56–65 | >66 |
|---|---|---|---|---|---|---|---|---|---|---|---|
| 1. B1 | 39 | 6 | 16 | 6 | 11 | 0 | 0 | 0 | 0 | 0 | 0 |
| 2 B5 | 30 | 0 | 0 | 0 | 2 | 6 | 1 | 5 | 4 | 10 | 2 |

TABLE 2
The average range and difference in age are listed below.

| Group: | Average age: | Range | Difference |
|---|---|---|---|
| 1 | 18.5 | 14–35 | 21 |
| 2. | 56.0 | 31–69 | 38 |

### As seen, the population of volunteers differs as far as age is concerned.

TABLE 3
Further characterization of the groups assigned to different creams.
The values are expressed in %:

Group:

| | B ®-1 | B ®-5 |
|---|---|---|
| Generalized Acne lesions; | 16.7 | 0 |
| Localized infected lesions | 83.3 | 0 |
| Previous treatment: | 23.3 | 80.0 |
| Menstruation effect: | 26.7 | 0 |

The data collected in table 3 shows that 16.7% of group B®-1 had generalized Acne lesions. It also provides information on how many volunteers' in each group ineffectively searched for dermatological and cosmetic help. There is quite a clear-cut difference between skin of younger and mature group. The number of participants that reported failure of the dermatological and cosmetic interventions increases with the age of volunteer. The activation of the inflammatory processes during menstruation period was observed only among group B®-1.

TABLE 4
The preexisting allergic reactions expressed in % values.

Group:

| Sensitivity to: | B ®-1 | B ®-5 |
|---|---|---|
| Alcoholic extracts | 30.0 | 0 |
| Water | 10.0 | 15.0 |
| Soaps | 33.3 | 10.0 |
| Varies cosmetics compounds | 11.0 | 65.0 |
| Pollen | 0 | 7.5 |
| Drugs/Antibiotics | 20.0 | 0 |
| Detergents | 0 | 37.5 |
| Food | 7.0 | 10.0 |

The younger participants reported more frequently sensitivity to soap, hard water, alcoholic extracts, and antibiotics. This may indicate more frequent use of these elements among young people. The older group reported sensitivity to detergent and cosmetics compounds. This information indicates existing problem with today's beauty products available on the market.

The information regarding skin type, complexion, and type of lesion was collected in the next table.

TABLE 5
Status of the skin before the Biovien ® product application:
the frequency was expressed in %

The group
tested:

| | B-1 | B-5 |
|---|---|---|
| The type of skin: | | |
| a. mixed | 24.0 | 50.0 |
| b. dry | 3.0 | 50.0 |
| c. oily | 73.0 | 0 |
| B. The skin complexion: | | |
| a. bright to yellow | 20.0 | 70.0 |
| b. gray | 40.3 | 0 |
| c. Dark | 39.7 | 35.0 |
| C. Vessels: | | |
| a. singular | 26.7 | 32.5 |
| b. numerous | 3.3 | 65.0 |
| c. absent | 70.0 | 2.0 |
| D. Wrinkles: | | |
| a. Superficial | 6.7 | 33.3 |
| c. deep | 0 | 50.0 |
| d. absent | 93.3 | 16.7 |
| E. Elasticity: | | |
| a. normal | 100.0 | 5.0 |
| b. slightly decreased | 0 | 17.5 |
| c. decreased | 0 | 77.5 |

Presented in table 5 information indicates different character of changes in the skin among young and older group.

TABLE 6
The volunteers skin status before Biovien ® cream
application expressed in %:

| Biovien Cream: | B ®-1 | B ®-5 |
|---|---|---|
| a. Acne | 10.0 | 2.5 |
| b. Skin discoloration | 20.0 | 96.5 |
| c. Comedo presence | 100.0 | 0 |
| d. Foci of inflammation | 100.0 | 0 |
| e. Hyper-keratosis | 3.0 | 2.5 |
| g. Enlarged sebum vessels | 100.0 | 0 |
| h. Pores enlargement | 40.0 | 0 |
| i. Scars | 9.9 | 0 |
| j. Piling centers (spats) | 3.0 | 2.5 |

The lesion characteristic for the oily skin appeared only among volunteers using anti acne B®-1 cream. Changes on the skin of group A was manifested by numerous infectious centers (61.5%) and acne scars were seen in 41% of participants. Discoloration do to acne was seen in about 20% of cases. The skin of the group B was dry (50%), The color of the skin was bright (52%), The volunteers had numerous vessels, wrinkles. The elasticity of the skin was diminished among studied volunteers of the group 5. The skin discoloration in this group was seen in 96.5% of volunteers.

TABLE 7
Supervisor's reports collected during the trial.

| | |
|---|---|
| | The creams applied on the skin penetrate in to the skin fast. |
| 1. | The creams effectively moisturized the skin. |
| 2. | They influence the skin condition during and after application. |
| 3. | The creams provided comfort after application |
| 4. | All volunteers participating in the program finished their 3 weeks |
| | treatment without any complaints. |

TABLE 8
Changes on the skin observed after termination of
the program expressed in %:

| Product induced: | Bovien ®-1 | Biovien ® 5 |
|---|---|---|
| Redness of the skin. | 0 | 0 |
| Itching. | 0 | 0 |
| Burning sensation. | 0 | 0 |
| Appearance of not desired effects. | 0 | 0 |
| Improvement of the moisture effect. | 7 | 97.5 |
| Decreased sensitivity to environmental | 13 | 72.5 |
| insult. | | |
| Diminishing of the scaling processes. | 7.0 | 65.0 |
| Brightening of the skin complexion. | 3.3 | 47.5 |
| Diminishing Acne. | 33.3 | N/A |
| Diminishing inflammation centers. | 49.6 | N/A |

Skin itching and burning sensation seen before the treatment started to diminish about 10 days after initiation of the program.

Discussion:

One of the goals of this study was to provide a series of products free of toxic and allergic substances. They were designed to eliminate a need for the use of additional products complicating procedures. We strongly believe that a natural complex of the active substances, delivered simultaneously topically and orally, will be the answer to healthy skin body and will substitute the fantasy products. Selected participants in the first group had severe skin infections, located on the surface of face, shoulders, back and chest. Earlier cosmetic and dermatological treatments performed on 28% of volunteers after short period of improvement failed. Sixteen participants included in this study have been on the dermatological program without visible effect. It was established that 25 out of 30 volunteers were also sensitive to previously used cosmetic products containing alcohol, soap and other cosmetic ingredients. These variety of skin conditions (at starting point), constituted an ideal material for testing allergic effect of the Biovien® series, that were focused on controlling damages of the free radicals. Volunteers of first group, in the majority of cases had oily skin, with sebaceous deposit-comedon (100%). The color of their skin was prevalently natural, bright (51.3%). Skin retains its elasticity and the wrinkles were absent. The differences between both groups are shown in Tables 1, 2, 3, 4, 5, 6.

The toxicity and allergenic activity of Biovien® products tested on human volunteers revealed that creams were safe. It is worth to mention that some individuals, outside the program, used the cream and body lotion for over the year without appearance of the allergic and adverse reactions or loosing beneficial effect.

The cosmetics presently on the market may activate allergies. Support for this observation was provided when testing the volunteers employed in this study. Close to 47% had allergy against different cosmetic product. This number increases with the age and reached 65% (Table 4) in the group involved in testing Biovien®-5. Furthermore, among tested volunteers were present also individuals who reported allergy to more than one cosmetic. Such peoples sensitive to many allergens are prone to development of new allergic reactions. The data provided in this report clearly indicates that none of the used products was toxic for any member of the cohort. Lack of allergic reaction, among tested volunteers, indicates that Biovien® products, directed against free radicals, are hypoallergenic and perhaps even making the host more resistant to allergic reaction. Support was provided by instructors supervising volunteers during these studies. They observed gradual diminishing the sensitivity of the volunteers to different impulses during experimental period.

Conclusion. Three weeks of application of Biovien®-1 and 5 on the volunteer's skin, revealed safety future of the tested product. The volunteers found that the Biovien® 1 and 5 were not toxic, and do not activate existing allergy nor induced allergic reactions. In addition number of participants noticed the rejuvenation of the skin.

### EXAMPLE 2

Summary. This report contains the results of testing six different Biovien® creams on human volunteers. The aim of these studies was to evaluate the effects different anti-oxidant complexes present in different creams have on the skin of people at different age. Generated information revealed that none of the anti-oxidant complexes tested induced an adverse reaction or was toxic. The products did not activate existing allergies or induce allergic reactions. All products protected the volunteer's skin from negative environmental impact, showing they were well adapted to the different age of the users. The most significant effects of the cream was observed among volunteers suffering because of oily skin and acne. The other participants, especially the mature group, also noticed several favorable reactions like soothing of itching, aching, pain, or dry skin feeling. Furthermore feeling of improved elasticity and firmness of the skin were noted by mature members of the testing group. They reported that the skin became deeply hydrated and “re-plumped” with life. The skin becomes again radiant and tone was revitalized. Key words: Free radicals, anti oxidants, scavenges, acute and chronic diseases, degenerative processes, anti acne formulas, antioxidant complexes, allergy, oily skin, acne.

Introduction. Evaluation of the skin care products existing on the market today leads to the conclusion, that there is a significant difference between existing knowledge and the philosophy of the producers. It is not clear whether the effects of cosmetics observed till now, were due to their true property or the placebo effect. The awkward idea of only feeding the skin through the outer skin layer causes induction of different types of allergy and the generation of free radicals (1). The paradigm developed postulates better control over the free radicals, as a key element of protection from germs. The Biovien®1-6 creams, Biovien® 1-6 body lotions and Biovien™ 1-5 dietary supplements to control the level of free radicals (generated by human body) during entire life spun. Complexes were formulated upon the latest scientific achievements that indicate the role of free radicals in the human body. The results of this study were not yet publish.

When constructing the anti-oxidant complexes, three classes of antioxidants were involved: First, those responsible for inhibition of the production processes of free radicals; second the neutralization of present free radicals and third modulating the intracellular modulation. The polypeptide binding the iron ions-Lactoferrin represents the first class of antioxidant. The second group consists of scavengers that neutralize free radicals and is represented by biologically active components present in: C.L.A., Seabuckthorn oil, Flax oil, flavonoids or beta carotene. The third class consists of scavengers that control the intracellular level of free radicals.

Focusing on the information presented above, the Biovien® product progression was developed. Their approach was different from the cosmetic products present on the market today. The broad spectrum of Biovien® products of different antioxidants are capable of inhibiting free radicals derived from different parts of the body, and from different metabolic levels. In addition, the antioxidant complexes must be adapted to the different age of the consumer's. The new products were constructed on “the reverse pyramid” principles. Several potential antioxidant candidates were selected among many having anti free radicals properties. Each candidate was reevaluated. Once they passed the criteria of safety, potency, and lack of toxicity, they were used for the preparation of our antioxidant complex (1). A gradually increased numbers of scavengers were added to the different series of Biovien® products. This strategy was adapted in order to efficiently control the increased number of free radicals seen with age. The available information indicated that the main target for free radicals is the Immune, Central Nervous, and Circulatory systems. The Immune defense system also called “a dispersed throughout the body” organ governs two major mechanisms; the innate and the specific immune systems. Studies were performed on volunteers to validate whether the Biovien® products provide an effective protection for the innate system, securing health of the host and extending quality of life. To support the claims concerning efficacy, the volunteers study utilized cosmetic criteria. They were designed to answer: whether Biovien® series is safe and did not induce allergy or activate allergic reactions (1) Contained in this report are the presented effects of different Biovien® creams on volunteers at different age. Final products were registered for the European Common Market after passing safety criteria like the sterility test; product verification test and stability. These results confirm that these products are not toxic and did not induce allergy or activate allergic reactions. The designed products are helping the host in maintaining homeostasis.

Materials and methods. Biovien® creams 1-6 were used for this study. Volunteers of different age, with different skin problems, were enrolled in this study. The volunteer group consisted of 177 volunteers, age 16-76 years. Each group received a designed product for solving particular problems. They were adjusted to the age and skin condition of the volunteers. Before initiation of the program, each person passed through an initial screening phase where a battery of evaluation tests assessed the volunteer's condition. The criteria selected were from programs used by the School of Cosmetology at the Medical University of Lodes. Each person included in the program was instructed on how to wash the skin with soap before applying the cream (2 times a day). Every volunteer was instructed on how to apply the tested material on the skin and had to learn how to verify effect of the product applied on the skin. The studies were carried for 10 weeks. The protocol applied in the study called for the evaluation of different cosmetic variables. Basic cosmetic information required is presented in table 1 (below).

Criteria classifying all Biovien® products used (B-1, B-2, B-3, B-4, B-5,-B-6). 1. How easy the cream is applied on the skin and how it penetrates the skin. 2. How effectively it moisturizes the skin. 3. How it influences the skin condition during and after application. 4. What is the comfort after application? Present information concerning the age distribution among groups of volunteers engaged in these studies in table 9 (below).

TABLE 9
The age distribution in different volunteer's subgroups testing Biovien ® products (the
abbreviations for Biovien-1 = B1 to Biovien 6 = B6, respectively) is shown:

| Group: | No | 14–20 | 21–25 | 26–30 | 31–35 | 36–40 | 41–45 | 46–50 | 51–55 | 56–65 | >66 |
|---|---|---|---|---|---|---|---|---|---|---|---|
| 1 Cream B1 | 30 | 24 | 5 | 1 | 0 | 0 | 0 | 0 | 0 | 0 | 0 |
| 2. Cream B2 | 39 | 6 | 16 | 6 | 9 | 2 | 0 | 0 | 0 | 0 | 0 |
| 3. Cream B3 | 21 | 0 | 0 | 0 | 2 | 2 | 8 | 5 | 4 | 0 | 0 |
| 4. Cream B4 | 24 | 0 | 0 | 0 | 1 | 0 | 1 | 6 | 6 | 11 | 0 |
| 5. Cream B5 | 23 | 0 | 0 | 1 | 0 | 6 | 5 | 8 | 3 | 0 | 0 |
| 6. Cream B6 | 40 | 0 | 0 | 0 | 2 | 0 | 1 | 5 | 7 | 18 | 7 |
| Total | 177 | 30 | 21 | 8 | 14 | 10 | 15 | 24 | 20 | 29 | 7 |

The 177 volunteers, both females and males were recruited for this study. The volunteers were assigned to six different sub groups called 1, 2, 3, 4, 5 and 6. The age and the size of these groups were not similar. The youngest consisted of 69 individuals and the older consisted of 108 volunteers. The extended demographic evaluation of the volunteer groups is shown in Table 10.

TABLE 10
The average age, range and difference in age within the group are listed.

| Group: | The average age: | The range | Difference within the group |
|---|---|---|---|
| 1 | 18.0 | 14–30 | 12 |
| 2. | 25.7 | 16–37 | 21 |
| 3 | 45.0 | 31–55 | 24 |
| 4. | 53.9 | 30–64 | 34 |
| 5. | 42.3 | 41–66 | 25 |
| 6. | 56.0 | 55–69 | 34 |

The range of age varies from 14 to 69 years. Within groups, the range of age is considerably smaller and varies between 12 to 34 years. This permits to better evaluate the values of the cream tested. Information presented in Table 11 provides evidence of planned heterogeneity of the volunteers' populations.

TABLE 11
Further characterization of the groups assigned to different creams.
Values expressed in %:

| | Group: | | | | | |
|---|---|---|---|---|---|---|
| | B ®-1 | B ®-2 | B ®-3 | B ®-4 | B ®-5 | B ®-6 |
| Generalized | 16.7 | 20.0 | 0 | 0 | 0 | 0 |
| lesions; | | | | | | |
| Localized | 83.3 | 80.0 | 0 | 0 | 0 | 0 |
| lesions | | | | | | |
| Previous | 23.3 | 56.0 | 56.0 | 68.0 | 87.0 | 80.0 |
| treatment: | | | | | | |
| Menstruation | 26.7 | 0 | 0 | 0 | 0 | 0 |
| effect: | | | | | | |

The data collected in table 11 (above) shows localization and type of lesions on the skin of participants, mainly in the younger groups suffering because of oily skin. It also provides evidence on how many volunteers ineffectively searched for dermatological and cosmetic help. The number of past failures of treatment increases with age. Collected variables stress the difference between younger and mature group. Only girls from the youngest groups reported a flair of the inflammatory symptoms during menstruation period, since only they have the inflammatory processes on the skin.

TABLE 12
The allergic profile of the volunteers before Biovien ® products
assessment. The expressed values in %.

| | Sensitivity to: in group: | | | | | |
|---|---|---|---|---|---|---|
| | B ®-1 | B ®-2 | B ®-3 | B ®-4 | B ®-5 | B ®-6 |
| Alcoholic | 23.3 | 30.8 | 0 | 0 | 0 | 0 |
| extracts | | | | | | |
| Water | 30.0 | 10.3 | 0 | 12.0 | 13.0 | 15.0 |
| Soaps | 33.3 | 33.3 | 6.6 | 12.0 | 13.0 | 10.0 |
| Cosmetics | 26.7 | 18.0 | 46.2 | 68.0 | 62.0 | 65.0 |
| Pollen | 0 | 0 | 2.6 | 12.0 | 8.7 | 7.5 |
| Drugs/ | 20.0 | 10.3 | 7.7 | 8.0 | 13.0 | 0 |
| Antibiotics | | | | | | |
| Detergents | 0 | 36.0 | 10.2 | 36.0 | 17.4 | 37.5 |
| Food | 20.0 | 5.0 | 2.5 | 4.0 | 8.7 | 10.0 |

The younger groups reported frequent allergy to soap, hard water, alcoholic extracts, and antibiotics. (see also report 1). The older groups reported sensitivity to detergents and cosmetics. The frequency of the last two variables increases with the age of the participant. Sensitivity to food, alcoholic extracts decreased with the age of participants. The skin characteristics of volunteers were collected in Table 13 (below).

TABLE 13
Status of the skin before the Biovien ® product application:
The frequency of variables was expressed in %.

| | The group tested: | | | | | |
|---|---|---|---|---|---|---|
| | B-1 | B-2 | B-3 | B-4 | B-5 | B-6 |
| A. The type e skin: | | | | | | |
| Mixed | 20.0 | 66.7 | 26.6 | 44.0 | 40.9 | 50.0 |
| Dry | 3.3 | 0 | 0 | 56.0 | 59.1 | 50.0 |
| Oily | 73.3 | 33.3 | 71.4 | 0 | 0 | 0 |
| B. Carnation of the skin: | | | | | | |
| Bright to yellow | 20.0 | 51.3 | 71.4 | 80.9 | 63.6 | 70.0 |
| Gray | 53.3 | 41.0 | 8.0 | 0 | 4.5 | 0 |
| Dark | 6.7 | 7.7 | 9.5 | 20.0 | 31.8 | 35.0 |
| C. Vessels: | | | | | | |
| Singular | 26.7 | 7.7 | 42.9 | 32.0 | 31.8 | 32.5 |
| Numerous | 3.3 | 7.7 | 38.3 | 56.0 | 68.2 | 65.0 |
| Absent | 70.0 | 84.6 | 19.0 | 12.0 | 0 | 2.0 |
| D. Wrinkles: | | | | | | |
| Superficial | 6.7 | 33.3 | 66.7 | 24.0 | 36.0 | 25.0 |
| Numerous | 0 | 0 | 0 | 20.0 | 41.0 | 30.0 |
| Deep | 0 | 0 | 28.5 | 52.0 | 21.0 | 45.0 |
| Absent | 93.3 | 66.7 | 4.8 | 4.0 | 2.0 | 0.0 |
| E. Elasticity: | | | | | | |
| Normal | 100.0 | 100.0 | 4.8 | 8.0 | 9.0 | 5.0 |
| Slightly decreased | 0 | 0 | 33.2 | 14.3 | 18.2 | 17.5 |
| Decreased | 0 | 0 | 62.0 | 77.7 | 72.8 | 77.5 |

Variables presented in Table 13 (above) indicate the skin changes have different character between young and older groups. The changes most frequently noticed were the skin type, wrinkles, elasticity of the skin, and the vessels formation. The lesions and other changes observed on the skin are listed in Table 14 (below).

TABLE 14
The complaints reported by the volunteers before the Biovien ® cream application.
(Expressed in %):

| | Biovien Cream: | | | | | |
|---|---|---|---|---|---|---|
| | B ®-1 | B ®-2 | B ®-3 | B ®-4 | B ®-5 | B ®-6 |
| a. Acne | 10.0 | 2.6 | 4.8 | 8.0 | 0.0 | 2.5 |
| b. Skin discoloration | 20.0 | 43.6 | 38.1 | 52.0 | 100.0 | 96.5 |
| c. Comedo presence | 100.0 | 64.1 | 0 | 0 | 0 | 0 |
| d. Foci of inflammation | 100.0 | 76.4 | 0 | 0 | 0 | 0 |
| e. Hyper-keratinosis | 50.0 | 7.7 | 0 | 0 | 4.5 | 2.5 |
| g. Enlarged sebum vessels | 100.0 | 72.0 | 0 | 0 | 0 | 0 |
| h. Pores enlargement | 40.0 | 69.2 | 4.8 | 0 | 0 | 0 |
| i. Scars | 43.3 | 41.0 | 0 | 0 | 0 | 0 |
| j. Piling centers | 20.0 | 7.7 | 14.3 | 8 | 0 | 2.5 |

The list of variables presented in table 14 showed that the Acne is distributed evenly among different groups of volunteers. This indicates a different etiology and patho-mechanism than was seen in the group with oily skin syndrome. The Oily skin disorder appeared only in B®-1 and B®-2 groups. Two variables from the acne disorder remain in group B®-3; the skin discoloration and the pores enlargement. The skin discoloration increases with the age of participants to the level where almost everybody was affected.

Results. The changes observed following 10 weeks of Biovien cream application are summarized below. Lack of signs of allergic reactions in regions of normal and affected skin bring the Biovien™-series in to a unique class of creams, permitting to combat different types of bacterial infections without induction of any adverse reactions and avoiding activation of allergic flares. In contrast, the changes observed, (Effects) noted in each tested group are summarized below.

Study 1. Effect of the Biovien™ 1 cream on Oily and Acne skin. In this study 30 volunteers of both sexes were enrolled. The group consists of 14-30 years old individuals. After initial selection, the allergic profile has been established and the skin changes were registered. All participants had severe skin infections, located on the surface of the face, shoulders, back and chest. Previous cosmetic and dermatological treatment failed for twenty-three percent of participants. It was also established that 26.7% of volunteers were sensitive to cosmetic products containing alcohol, soap and other.

It was found that: (1) The signs of inflammation were diminished after 10 weeks application of Biovien®-1 and 2 cream. (2) Number of black heads (markers of the oily skin) was diminished. (3) The excessive sebum secretion was diminishes to normal level. (4) The skin brightness was enhanced and epithelial zone was better moisturized. At the same time, the thickness of the corneal layer was diminished. (5) Brighten were post acne discolorations of the skin. (6) Diminishing pore size was noticed. Based on the presented effects, the Biovien®-1 cream can be considered as an anti bacterial that acts best prevent the appearance of blemishes.

Study 2. Effects of the Biovien® 2 cream on skin infections. Group of 39 volunteers of both sexes was enrolled in this study. The group consisted of participants 14-40 years old. The volunteers described for using B®-1 cream only had lesions distributed not only on the skin of the face, but also on the skin of neck and shoulders. Fifty six percent of volunteers treated earlier by cosmetic and dermatological methods that were based upon aseptic and antibiotic regiments failed after a short period of improvement. Thirty three percent of volunteers were sensitive to soap. Over 30% were sensitive to alcohol extracts and 36% to detergents. After 10 weeks of the Biovien®-2 applications mentioned, changes greatly diminished or disappeared. Based on presented information, the Biovien®2 creams have an anti-acne and anti-inflammatory property.

Study 3. Evaluation the efficacy of the Biovien®-3 cream on adult skin. 21 volunteers participated in this study. They were selected from both sexes, and age range was 31-55 years. After initial examination, the skin problem was identified and an allergic profile had been established. About 38% of participants exhibited skin discoloration and 14% had accentuated piling centers. Part of participants (46%), prior to initiation of the study reported signs of allergy. All participants had advanced skin damages manifested by dryness, diminished elasticity and tightness of the skin, and presence of wrinkles around the mouth and eyes. Many participants had small but visible extended blood vessels. Part of volunteers exhibited discoloration of the skin.

Earlier treatments carried out by dermatologists or cosmetologist provided only temporary improvements. 1. After termination of the program, participant stressed a lack of adverse reaction and no development of new allergies. 2. Those with allergy to different agents indicated a lack of activation of an existing allergy. Based upon collected information the Biovien.®-3 cream can be classified as classical maintenance cream protecting the skin against sun damage.

Study 4. Evaluation of the efficacy of Biovien®-4 cream on mature skin was done on 24 volunteers of both sexes. They were 30-60 years old. After initial examination, the allergic profile and the actual status of the skin had been established. About 68% of participants, before initiation of the studies, reported they had adverse reactions to different cosmetics; creams, tonics, body milks, perfumes, etc. Among them were: Arnica, soaps, collagen, and peptides isolated from silk moth larvae, different preparations from honey, and chamomile extract and 36% of participants to various detergents. 56% had dry skin and 77% had decreased elasticity and tightness of the skin. Fifty two percent exhibited discoloration of the skin. Wrinkles around mouth and eye were present in all participants of this study. In many occasions, participants had small visible extended blood vessels and redness of the skin. Zones of keratin and peeling spots were well marked.

Earlier dermatologists or cosmetologists treatments reported by 68% of the participants provided only temporary improvements. 1. After ten weeks of the program, all participants reported beneficial effects of applied cream, such as better-hydrated skin and resistant to sun damaging effects. 2. Some reported diminishing pain and aching of the body. Based upon collected information the Biovien®-4 cream can be considered as a regenerating agent.

Study 5. Evaluation of the efficacy of the Biovien™-5 and 6 creams, on adult skins. This study was conducted on 13 and 40 volunteers of both sexes, 35-70 years old. The two groups were combined, because biologically active elements present in the creams were identical, only their proportion was different. After initial examination of the allergic profile, the actual status of the skin had been established. All participants had signs of various advanced skin damages, manifested by wrinkles, dryness, blemishes, diminished elasticity and tightness of the skin. Elasticity of the skin for B-5 group was decreased in 72.8%, whereas in group 6 was decreased in 77.5%. Discoloration for both groups was in the range of 50% and dry skin was reported in about 50% of the participants for both groups. In many occasions, participants had small visible extended blood vessels and redness of the skin. Zones of keratin and peeling of the (dry) skin in both groups were well marked out. Presence of wrinkles around the mouth and eyes were noted in participants of this study. (B-5 was 92% and B-6 100%). Earlier dermatologist or cosmetologist interventions provided only temporary improvements. Part of the participants reported allergy to different medication and cosmetics like creams, tonics, body lotions and antibiotics. (See table 12, above) The identified allergens were; Arnica, soap, detergents, beauty products with collagen, honey, chamomile, and peptides isolated from silk moth larvae, lanolin, and color beauty product. In addition participants reported sensitivity to cold, windy weather, and sun. Both groups exhibited similar patterns as far as the skin changes are concerned.

The supervisor's of the Biovien 5 and 6 creams groups noticed that: (1) The cream was easily applied and penetrates in to the skin, without leaving a greasy filling; (2) Helped the skin to retained moisture; (3) Improved the skin elasticity; (4) Diminished the discoloration, betters the skin color: the skin becomes definitively bright and healthy looking; (5) Wrinkles diminished and become less deep than before treatment; (6) Skin regain it smooth, youthful look; and (7) Zones of the dry skin significantly diminished or totally disappeared.

After 10 weeks of participation in the study, members of the both groups Biovien®-5 cream and Biovien®-6 cream reported: (1). Improved skin hydration, and vitality; (2) The skin elasticity was noticed; (3) The skin tension increased; (4) Dry skin feeling diminish, disperse hyper-keratic area were shrunken; (5) The redness of the dry skin significantly diminished or totally disappeared; (6) The skin become smoother, wrinkles definitively diminished and skin become more resistant to atmospheric stress; (7) Those participants who reported burning and pain sensation before initiation of the study noticed, that symptoms started to disappear about 10 days after initiation of the program. Some volunteers who had arthritis pain before initiation of the program notices release, especially those who used B®-6. (This observations need further confirmation); (8) Skin becomes more resistant to atmospheric stress, protected from harmful environment and sunlight; (9) After cessation of treatment the signs of (noticed reactions) damages did not reoccur; and (10) All participants finished their 10 weeks of treatment without any complaints.

After ten weeks of the application of Biovien®-1-6, effect was manifested not only in above statements, but also by slowing down the aging process and the appearance of rejuvenated-regenerated skin. Based on observation both creams can be recommended as an anti-wrinkle, intensively regenerating agent especially for dry and severely damaged skin. The search for adverse reactions among volunteers using creams: B®-1, B®-2, B®-3, B®-4, B®-5, B®-6 provided the following results summarized in table 16 (below).

TABLE 16
The results from study for toxic and adverse reactions and
for allergic responses are summarized below:

| | No | |
|---|---|---|
| | observations | Results |
| Redness of the skin B ®-1–B ®-6 groups | 177 | 0 |
| Aching of the skin B ®-1–B ®-6 | 177 | 0 |
| Burning sensation of the skin B ®-1–B ®-6 | 177 | 0 |
| Other adverse reactions of the skin B ®-1–B ®-6 | 177 | 0 |

Results presented in table 16 clearly indicate that Biovien® creams have a nonnegative impact on redness, burning, itching sensations of the skin.

Skin evaluation was performed before, during, and after treatment termination (10 weeks). Instructors before and during the trial collected information on the below variables. Lack of adverse reactions during applications of Biovien® products were verified by an observation of the absence of signs of inflammation or allergy. All participants finished their 10 weeks treatment without complaints. The effect of Biovien®-cream are shown in table 17.

TABLE 17
Other effects of the creams observed by participants during pilot trial.

| Type of product | All Biovien ®- |
|---|---|
| Elasticity of the skin improved | 97.5 |
| Diminished sensitivity to environmental factors observed. | 72.5 |
| Decrease keratinization on the skin noticed. | 65.0 |
| Diminishing wrinkle formations seen. | 67.0 |
| Blood vessel size decrease and discoloration occurred. | 47.5 |

Discussion. The effect of the Biovien® series of products to antioxidants adapted to the age of user was determined. The design of Biovien® was done after thorough scientific evaluation of the cosmetic field and the identification of problems connected with the market. This custom-made approach and concentration against free radicals warranted great success. As has been shown in Example 1 (above), products were not toxic and did not generate allergic reactions nor activate existing allergies. In present studies, the effect of Biovien® products performed on a larger group of volunteers, confirmed initial observations. The very young group with oily skin and acne and older with marked signs of skin aging differ from each other in many variables. The differences were shown in tables: 1, 2, 3, 4, 5, 6 and 7.

The Biovien® creams 1 and 5, used in Example 1 (above), were adsorbed fast without induction of an adverse reaction. These observations were confirmed in this Example. It is worth to mention that some individuals, outside the program, reported that even after a year of using the cream, they did not experience any allergic and adverse reactions or loose the beneficial effect of Biovien® cream.

The presented studies underlined the role of several selected biologically active compounds: Lactoferrin cooperating with the innate system, protecting it by binding iron ion (3,4). When the Iron ions are liberated or excreted through the skin, it undergoes further oxidation called Fenton reaction (1). During this oxidation process, iron utilizes the host's carbohydrates. The cleavage of the carbohydrates leads to the release of large quantities of hydroxides (OH) free radicals and a large quantity of free energy and damage to the skin sceleton. The damages to the skin are utilized by the germs, living as a sapropytes on the skin surface and inside the body. Their invasion in to the skin and whole organism damages further the skin ultra-structure, causing wrinkle formation. Released hydroxides are responsible, among other for changes of the skin pH and accelerate the skin damage. Among many known innate mechanisms, the inhibitory complex protecting the host against invading agent is playing the key role as the first line of defense.

The other system involved in protection of the host from germ invasion is the interferon family. The Interferon family includes 14 species marked in the Greek's alphabet; α, β, γ, δ, ε, ζ, η, θ, ι, κ, λ, μ, ν, π. In each Interferon type, at least one member represents the species, but in the alpha group, present are 15 different members called Human IFN α: a, b, c and so on. On top of that, genetic engineering technology permitted to create recombinant Interferon hybrids made by fusion of two genes within a family. They carry the name pegylated Interferon. (13). They provided better anti germs properties in vitro studies. (In vivo), like others, are inducing production of antibodies when they are used in therapeutic doses. Different Interferon species support each other, forming two information networks called “Interferon's cascade”. Interferon cascade I activates reactions inside the cell, rendering cell resistant to invading germs of not necessary viral origin. This cascade explained why interferon protects, not only against viruses, but other germs as well.

The second cascade spreads the information of incoming invasion to all host cells of the human body. The instruction on how to protect cells against invasion is spread further from one cell to another by activation of different secondary interferon species and activating additional mechanism transferring the information from cell to cell present in close contact. Both mechanisms are rendering cells resistant to germ infection. Both have an amplification character. The Interferon cascade, not interrupted, is capable, through a series of amplification steps to provide information to all 10¹⁷ cells forming the host. The cascade II limits multiplication capability of the invading germs. All above-mentioned defense mechanisms are very sensitive to the free radical action. This explains the failure of the innate system in protecting the host and controlling the germ invasion. The specific immune responses are activated at the end of infection and provide little help in the initial phase of the fight against invading germs. The immune mechanisms are protecting the host against the return of the same germ. Those defense mechanisms are further supported by the lactoferrin. Both Interferon and lactoferrin control the free radicals synthesis (unpublished information). The anti microbial non-specific barriers created by the above described innate system are further supported by a group of chaperons controlling metabolic processes inside the cells.

Finally the anti-oxy-vitamin complex, formed by polyfenoles, antocyanes, flavonoids, retinoids, licopensand beta carotene, has the capability to neutralize the free radicals, protecting the innate system and preventing the formation of oxidative stress and synthesis of the 4 Hydroxyl Nonenal (4 HNE)(23, 24). The last compound is made by the host in emergency situations during a time when the concentration of free radicals is so high that oxidative stress reactions start to initiate a series of killing host reactions. One process affected by 4HNE is programmed cell death called “apoptosis”. The 4HNE, by itself is the free radical. However it reduces the number of circulating free radicals made during germ infection. The 4 HNE can bind practically to every macromolecule present in the body, causing metabolic havoc. Among most damaging reactions is the activation of apoptosis, (The programmed cell death) since it initiates a variety of forms of chronic diseases. The role of the flax seed extract on the skin and the health of human beings has been known for ages. At the time of the Roman Empire, one of the Roman emperor ordered his soldiers to use Flax seed oil daily to keep healthy and strong. Similar stories hold true for Hyppohae ramnoides oil. It was used by soldiers of Mongolian Emperors for ages to maintain the health and strength of their horses. As a result of the combined efforts of lactoferrin, chaperons and anti-oxy vitamin, the innate system is effectively protecting the host, forming a barrier for invading germs. This obstacle provides needed time, for the immune system to generate specific defenses. This action leads to further control of frees radicals. The described above anti germ mechanisms are further supported by the non and specific arm of cellular immunity (leukocytes, macrophages and NK cells). The complete power of the innate system can be appreciated by the fact that it controls, in lumen of the intestinal tract, permanently thriving circa 2 kg of germs.

Since the host is not capable to synthesized anti-oxy vitamins and other scavengers, they have to be provided from outside sources. Complexes of antioxidants used herein include: chaperones, the anti-oxy-vitamins, antioxidants inhibiting production and neutralizing free radicals. The elements present in these complexes become essential in maintaining a healthy host.

The term “germ” covers different species of Bacteria, Molds, Yeast, Mycoplasmas, Rickettsiae, Legionalla, Chlamydia, Viruses, and Prions. In addition to this group, can be classified mono-cellular parasites like Malaria, Leishmania or Toxoplasma. When they attack the host they also release quantities of free radicals. During host invasion, the germs affect the host metabolism by induction synthesis of the free radicals. Every germ is inhibiting the host metabolism at its different level and therefore many different types of anti free radicals needs to be involved in defense of the host. When germs are not opposed by host defenses, they multiply very fast. Within twenty-four hours they can generate so many progenies, that they can affect every cell of the host body.

Based upon the generated information, it is possible to recommend the Biovien series in following situations: (1) The Biovien®-1 cream can be recommended as an antibacterial formulation for skin prone to the infections. It can be considered as an anti bacterial cream that acts even before a blemish appears. (2) The Biovien®-2 cream can be recommended to be used as an antibacterial and anti free radical for oily skin infected with germ inducing acne. (3) The Biovien®-3 cream can be recommended to be use as a protective and anti-wrinkle cream. (4) Biovien®-4 Cream, represents a unique class of creams that have the ability to slow or diminish the aging process. (5) The Biovien®-5 cream can be recommended as an intensively regenerating agent, especially for severely damaged and dry skin. This cream can be also considered as actively inhibiting the aging processes. (6) The Biovien®-6 cream can be used as an intensively regenerating agent.

The cosmetics presently on the market did not prevent induction of the allergies. After 10 weeks of application in our own studies on volunteers, no allergic reactions were noticed. Those volunteers had allergies directed against different cosmetic products at the beginning of the study. This observation found further support by the group of volunteers who already were allergic to previously used cosmetics (sensitivity varied from 18-68%). Also, those people in the Volunteers group, having prior problems with allergy, did not notice a flare of the allergic reaction after Biovien® application. (These observations concerning the skin conditions, constituted an ideal situation for testing Biovien® series and evaluating the effect of products on the control of free radicals). In contrast to previous experience, they noticed signs of diminishing sensitivity to allergen. This observation, suggest that it is possible to desensitize people. Furthermore, some of tested volunteers were allergic to more than one cosmetic. The sensitivity increases with the increased age of the volunteers, reaching up to 68% among the oldest. It is important to notice, that failure to induce allergy among those volunteers indicates that Biovien® products are hypoallergenic. These results represent significant findings even though they were recorded only by cosmetic criteria. Lack of allergic reaction, among tested volunteers, indicates that Biovien® products, directed against free radicals, are hypoallergenic, since the allergic group is particularly prone to the development of new allergies. The affect on allergy found additional support from instructors that supervised volunteers during these studies. They did not observe any hypersensitive reactions among tested volunteers during the experimental period of time. Furthermore, they noticed that number of allergic reactions subsided. This observation found further support in the observation that the inflammatory processes seen among the youngest participants also significantly diminished after the application of Biovien®-1 and 2. The disappearance of the inflammatory processes lead to the observation that inflammation or allergic reactions do not affect the vesicle formation since the blood vesicles appear even more frequently among older people with dry skin.

Presented in table 5 information indicates the skin changes have different character in young and older groups. The most frequently differences occurring in the skin type are the wrinkles and elasticity of the skin. The pilot studies (1) and data from this report supplement each other in recognizing that the products used were not toxic. One significant difference seen in both studies between the skin was the change of its appearance. This was probably due to the extended time of application in this study. Normally the number of wrinkles increases with the age of tested population. They might represent signs of incoming time when concern about aging has to be addressed. Partial disappearance among tested or the loss of their intensity needs further confirmation. Elasticity of the skin gradually decreases with the age of the participants; reversal of this sign has the same significance as wrinkle formation.

Other properties worth mentioning is the evidence that Biovien® creams were well adapted to the age of the user. There are very few cosmetics on the market that will exhibit similar properties. This report indicates that in contrast to Biovien®, the ordinary cosmetics fail to satisfy consumers. The number of failures measured by increasing allergic reactions represents an objective observation.

Presented evidence demonstrates that products designed by the present inventors to be not toxic, to not activate existing allergies, nor to induce new allergies. This success was achieved by including natural components, known for ages as harmless and useful and having antioxidant and anti allergic property. Those facts place the Biovien® series in unique position among present skin care products.

### EXAMPLE 3

Effect of the topical and oral application of Biovien®-1 on oily skin with acne. The aim of this study was the development of a line of cosmetics and dietary supplements adapted to specific needs of skin at the different age of the host. The skin appearance reflects the metabolic status of the whole body. To maintain this status, living organism requires a constant supply of basic materials like proteins, carbohydrates, fats, vitamins, minerals and biologically active elements. Certain elements of the food can be delivered in multiple forms; others have to be supplied in a more restricted manner. Breaking the rules governing physiological requirements leads to the development of a series of dysfunctions, affecting the comfort of living and life spun. They are also called chronic disorders. Atherosclerosis, diabetes, or HIV may serve as an example. Presented in this report, are the results of clinical studies using Biovien®-1 alone and in conjunction with Biovien™ dietary supplement. Studies revealed that the anti-oxidative complex administered simultaneously in the form of cream and dietary supplement provides better control of oily skin and acne than a single cream application. Combination of Biovien® 1-6 cream with the Biovien™ 1-5 dietary supplement has amplification property.

Introduction. It was found that the complexes of cream and dietary supplements support the body and the skin, better controlling the free radicals than a single cream. Natural biologically active substances were used simultaneously and delivered topically and orally to the body. It was found that the damaging effect of free radicals are also responsible for conditions like oily skin and acne. Since production of free radicals occurs from the beginning of human life and intensifies with age, the control, prevention, and/or correction are essential during the human life spun.

The oily skin condition, especially when it is complicated by bacteria infection, plays a significant role among many skin problems affecting human population at different age. Unfortunately, very little progress has been achieved in solving the oily skin problems up till now. The significance and benefit of this approach, was shown in the evaluation studies on volunteers. The Medical University of Lodz Poland carried out the studies. (1) They showed that Biovien® products were not toxic and did not induced or activate existing allergy. In second study (2) volunteers assessed the Biovien® value as protectors of the body and the skin against damage by the free radicals. This study proved that significant protection against damages of the free radicals was achieved. The present report (3) provides evidence that the Biovien® 1 products given simultaneously on oily skin with acne and orally works more effectively than application of a single cream. The results show that the combination shortens the period of time, between beginning of application and effect seen on oily skin. The support has an amplification character and demonstrates the importance of the phenomenon in general. It appears that customized approach, according to age and particular needs, directed towards simultaneous delivery of needed nutrients, for control of free radicals to the skin and the whole body, is the right approach.

Material and methods. Groups of people with oily skin condition and acne were selected for these studies. The testing was carried out on 69 volunteers selected from a group of young people with oily skin and chronic acne infections. The lesions were prevalently localized on the facial part of the skin, but frequently were also observed in other areas of the body. Different cosmetologist and dermatologist previously unsuccessfully treated the participants before initiation of the Biovien studies. The study was conducted in the School of Cosmetology of the Medical University of [symbol] ód\hat{z}, Poland.

Before initiation of the Biovien®-1 trial, each participant's history of oily skin and acne conditions were recorded. Trained instructors, familiar with this disorder evaluated the skin, type of lesions, and the frequency of its appearance. The variables were recorded according to standardized protocol developed at the Medical University of Lodz, Poland. Each participant was instructed how to evaluate results of the product applications and how to assess the effect of treatment by himself or herself.

The following variables were assigned for each participant: A. How easily cream was dispersed on the surface of the skin?; B. How fast Biovien®-1 cream was adsorbed and the moisturizing effect noticed; C. Effect on the systematic application on the skin was also tested; D. How comfortable the volunteer will feel after the use of the cream?; and E. Whether volunteer observed undesired effects during the testing period like: itching, piling, burning, or tingling of the skin. All participants were divided into two groups; one consisted of 39 volunteers received Biovien® 1-cream, applied twice a day on the face skin.

The second group consisted of 30 volunteers applying the cream in similar fashion as the first group; in addition they received Biovien™-1 dietary supplement delivered orally, twice a day. Before the first application, the participants were informed, how to clean their skin with soap and to wash with germ free water once a weak each participant had a facial mask made from the same Biovien®-1 cream. The same procedure was applied to all 69 participants for a period of 10 weeks. After that period the final evaluation was conducted. Three and six months after termination of the program, the participants were asked to return into the School of Cosmetology, to evaluate the late effects of the trial.

Results: Distribution of the volunteers according to their age is shown in table 18 (below).

TABLE 18
The age distribution of the participants:

| Age: Group | 11–15 | 16–20 | 21–25 | 26–30 | >31 |
|---|---|---|---|---|---|
| Group 1: 39 | 0 (%) | 6 (15.4%) | 16 (41%) | 6 (15.4%) | 11 (28.2%) |
| Group 2: 30 | 8 (26.7%) | 14 (46.7%) | 7 (23.3%) | 1 (3.33%) | 0 (%) |

Both groups were not identical. The severity of condition underlined the fact that the second group of volunteers was much younger than in group 1. The health skin status of the selected participants before testing is summarized in the next table.

TABLE 19
Observed skin changes:

The group tested:

| | B-1 | B-2 |
|---|---|---|
| Participant No. | 39 | 30 |
| The type e skin: | | |
| d. Mixed | 20.0 | 66.7 |
| e. Dry | 3.3 | 0 |
| f. Oily | 73.3 | 33.3 |
| B. Complexion of the skin: | | |
| c. bright to yellow | 20.0 | 51.3 |
| d. gray | 53.3 | 41.0 |
| c. dark | 6.7 | 7.7 |
| C. Vessels: | | |
| d. singular | 26.7 | 7.7 |
| e. numerous | 3.3 | 7.7 |
| f. absent | 70.0 | 84.6 |
| D. Wrinkles: | | |
| a. Superficial | 6.7 | 33.3 |
| b. Numerous | 0 | 0 |
| c. deep | 0 | 0 |
| d. Absent | 93.3 | 66.7 |
| E. Elasticity: | | |
| a. Normal | 100.0 | 100.0 |
| b. Slightly decreased | 0 | 0 |
| c. Decreased | 0 | 0 |

TABLE 20
Comparison of the basic variables among two volunteers groups

| | Group 1 | | Group 2 | |
|---|---|---|---|---|
| Tested | 39 | % | 30 | % |
| Nest eggs occurrence | 30 | (75.0) | 30 | (100.0) |
| Skin inflammation centers | 8 | (20.5) | 23 | (77.3) |
| Tendency to skin inflammation | 23 | (59.0) | 7 | (23.3) |
| Pores enlargement | 28 | (72.0) | 15 | (50.0) |
| Skin hyper-keratosis and piling | 6 | (15.0) | 15 | (50.0) |
| Discoloration | 14 | (36.0) | 12 | (40.0) |
| Scars | 15 | (38.0) | 15 | (50.0) |

Conditions observed in-group 1 and 2 indicate that the volunteers in-group 2 have a more severe form of oily skin disorder. The inflammation and hyper-keratosis variables were particularly well seen. Adverse reactions to cream applications are shown in next table.

TABLE 21
Lack of the adverse reactions measured during cream Biovien ®-1
application and combination cream and Biovien ™-1 dietary supplements:

| | | |
|---|---|---|
| Clinical signs: | Group 1 | Group 2 |

| Tested | 39 | % | 30 | % |
|---|---|---|---|---|
| Appearance of redness | 0/39 | 0 | 0/30 | 0 |
| Aching | 0/39 | 0 | 0/30 | 0 |
| Other | 0/39 | 0 | 0/30 | 0 |
| Burning sensation | 0/39 | 0 | 0/30 | 0 |
| Other signs of allergy | 0/39 | 0 | 0/30 | 0 |

Accumulated information, during the 10 weeks of testing, indicates that the Biovien®-1 cream alone and Biovien™-1 dietary supplements gives no adverse reactions. This indicates that application of both cream and Biovien™-1 dietary supplement simultaneously is safe and does not induce adverse reactions. Application of such combination to volunteers with recorded allergy, did not affect or activate allergic reactions

TABLE 22
Effects of applications Biovien ®-1 cream alone and combination
with Biovien ™-1: Reduction of the observed variables.

| | | |
|---|---|---|
| Symptoms: | Group 1 | Group 2 |

| Tested. | 39 | % | 30 | % |
|---|---|---|---|---|
| Oil excretion. | 30/3 | 92.3 | 27/3 | 92.3 |
| Nest eggs | 30/8 | 73.4 | 27/8 | 70.0 |
| Acne | 31/4 | 87.1 | 29/0 | 100.0 |
| Carnation changes | 14/0 | 100.0 | 9/0 | 100.0 |
| Pore size | 28/0 | 100.0 | 13/5 | 61.5 |
| Discoloration | 14/5 | 64.3 | 9/0 | 100.0 |
| Scars | 15/8 | 53.4 | 13/0 | 100.0 |

Application of cream or cream with dietary capsules caused different reactions on the skin of volunteers. As may be seen from table 5, several variables disappeared or were greatly reduced during 10 weeks of application. The reduction of variables was observed at the end of the application and was more noted in the group using a combination of cream and dietary supplements. Kinetic of the acne disappearance is found in Table 23 (below).

TABLE 23
Kinetic of skin responses to Biovien ®-1 alone or combination
with Biovien ™-1 applications as far as acne is concerned.

| | Group 1. | | Group 2 | |
|---|---|---|---|---|
| | 31 | | 29 | |
| Period of time: Responders: | responders; | 79.5% | responders | 96.7% |
| 10–15 days | 0 | 0 | 18 | 62.1 |
| 16–20 days | 0 | 0 | 6 | 20.7 |
| 21–56 days | 12 | 38.7 | 5 | 17.2 |
| 57–70 days | 19 | 63.3 | 0 | 0 |

Data presented in table 5 and 6 demonstrates that simultaneous application of the Biovien®-1 cream with Biovien™ dietary supplements shortens the period of time between the application and the appearance of skin responses. The number of successful acne eliminations is also in favor of combination application. These objective changes, clearly demonstrate the appearance of the amplification reaction. Disappearance of aching present before initiation of the study generally disappears faster in the group of combination application than in the group using the cream alone. Inflammatory processes in oily skin disorder are healed through the shortening of the exudation phase of inflammation and the healing occurs without scares formation. Although both group were not necessarily identical, never the less, results clearly demonstrate, that the combined application gives better results.

Discussion. The selected volunteers for comparative study has similar age, signs of oily skin, and skin involvement to the group described in Examples 1 and 2. Oily skin and acne disorder are conditions in which the skin changes called oily skin and acne frequently occur during the maturation period, at a time of hormonal unbalance, leading to erroneous responses to the signals. The sensitivity to infection is particularly increased due to overproduction of the skin lubricant, which is a “good nutrient” for saprophytic bacterial flora. Furthermore, the oily skin condition is accompanied with overproduction of sweat. The sweat, in turn carries the iron ions that are essential elements for growth of skin saprophytic bacteria. These two, the oily skin and excess of iron, creates favorable conditions for skin's bacteria to thrive within the comedon. Comedo is called acne when infected with bacteria present on the skin. The growth especially occurs in ducts of the glands producing the skin lubricants. As a control, acne skin was treated with alcohol, salicylic acid or desquamation or peeling the epidermal part of the skin agents. Procedures rather harmful to the skin especially used for prolong time. The microbial flora mentioned above is extremely difficult to control, since germs frequently penetrate in to different parts of the skin, through the skin channels, that are difficult to penetrate by drug or antibiotics. The exposed germs may remain undisturbed until they become resistant to drug or antibiotics and other medicaments.

Since oily skin disorder is accompanied by an elevated level of free radicals, the introduction of new products delivering the anti oxidant complexes were provided. In addition, the bacterial flora present in acne lesions represents a group of normal saprofitic bacteria normally not pathogenic for human. Those germs are frequently dependent from the iron ion. Iron is an essential element of life for them. The fact that in the acne process, the invading germs are recruited from normally growing saprophytic flora of the skin supports this thesis. The permeability of the upper layer of the skin is limited, for that reason, it is necessary to deliver active compounds by both routs, topically and orally. The idea of simultaneous introduction of the same active compound in Biovien® 1-6 creams, Biovien body lotion and dietary supplement disclosed herein. This new approach improves not only the skin conditions, but also protects the whole organism from damages of free radicals, in various age stages. In this report we provide evidence that simultaneous application of corrective doses of the biologically active compounds to the whole body and on the top of the skin are more efficient than application of one element.

Results indicate that simultaneous application of the same biologically active elements induces an amplification phenomenon. The amplification phenomenon was observed for the first time in Interferon interactions (3,4). It was later described that the mechanism transferring information from cell to cell also operates on the amplification principles (3,4). Demonstration of the similar phenomenon occurring in the correcting of metabolic processes leads us to believe that the amplification phenomenon represents a new, not yet fully recognized phenomenon. This placed Biovien® and Biovien™ series in to a new category of nutrigenic property, distinct from current existing cosmetic products on the market. According to the presently developed model, the designed series of Biovien® cosmetics Biovien® and methods of application represent a new class of products that may be called the probiotics. They address the need of the population at different age, for biologically active compounds. The body requires different types of scavengers and probiotic complexes at different life stages. Using this new approach, the reverse pyramid strategy for production of Biovien® creams, Biovien® body lotion, and Biovien™ dietary supplements gains a new significance. Examples 1, and 2 show that Biovien® products after prolonged application efficiently eliminated juvenile metabolic unbalance and acne. Using this amplification modality (the combination treatment) we shortened the period between application and appearance of the expected results. We strongly believe that a Biovien® and Biovien™ are physiological products, to control skin infections.

Created paradigm explains the mechanism of action for the biological active components; the Biovien® creams and dietary supplements. The paradigm assumes that the principal functions of the skin include protection, excretion, secretion, absorption, thermoregulation, sensory perception and regulation of immune processes, are all affected by free radicals. Simultaneous delivery of natural active principals, activates an amplification reaction that is not toxic and does not induce adverse reactions like inflammatory or allergy. The amplification reaction play an essential role within the body and ignoring this mechanism may lead to serious sequels or consequences.

The oily skin problem causes tremendous discomfort and psychological stress to affected young people. Only a low number of “victims” are sensitive to lengthy classical therapy of several months of systematic application before same therapeutic results could be seen. The approach disclosed herein corrects this condition, even when the skin of the whole body was affected.

### EXAMPLE 4

Effect of topical application of the Biovien®-products on the skin. Objective assessment on pH of the volunteer's skin.

Summary. Evaluation of the skin care products existing on the today market leads to the conclusion, that there is a significant difference between existing knowledge and philosophy of the producers. It is not clear whether claimed effects of cosmetics are due to their true property or the placebo effect. The awkward idea of feeding skin through the outside, using surfactants to force penetration of antigens present in the creams, certainly leads to induction of antibodies, allergies and adverse reactions, and generation of excessive quantities of free radicals. Those effects are particularly well seen among people using cosmetic from various producers to combat chronic inflammatory conditions of the skin called popularly, acne.

The Biovien® series of composition were created as disclosed herein to provide the human with natural and essential products for host life. They were made according to new theory. The Biovien® products were directed against the free radicals to prevent damages generated by them during the human life spun. Since the free radicals production increases with the human age, the Biovien® anti free radicals complexes were adapted to different human age. This goal was achieved by increasing scavenger potency. The lack of understanding the role of free radicals and their significance in human life leads existing anti oxidant efforts in to dead end and wrong conclusions. Simply ignoring the fact that the mechanism of free radicals action is behind changes of the skin and aging of the body and thus leads to amplification of their effect. Several reports utilizing faulty combinations of the antioxidants leads to generation of quite astonishing results, like acceleration of human aging.

To establish the role of antioxidants in human life, compositions and methods disclosed herein were testes under both subjective and objective criteria to evaluate changes created by the free-radicals in skin. Among many objective measurements selected for this study included: the skin pH, level of moisture, and lubrication. The study results presented in this report provided statistically significant evidence that creams and body lotions affect volunteers' skin by inducing normalization of skin pH, and modulation of the skin moisture and lubrication. Those changes provided evidence that the designed antioxidant complexes control the free radicals present, not only on the surface of the skin, but also inside the host body.

Introduction. Examples 1, 2 and 3 (above) provided evidence the Biovien products, tested on volunteers, are not toxic and did not induce signs of inflammation, or allergic reactions. Furthermore, tested Biovien® product did not affect already existing allergies. It was found that the elimination or control of free radicals in the host will modulate and control not desired reactions occurring, that shorten the life. Described herein in conjunction with products named Biovien®, cosmetics were developed and tested that focus on the control of free radicals responsible for aging and shortening life span. After evaluation of groups of individual scavengers, the scientist designed the antioxidant complexes. The complexes were based on existing natural products, having scavenger property, especially when overproduction of free radicals becomes a threat to the well being of the host. An additional condition for acceptance was that single elements in the designed complexes did not interfere with each other.

The skin is the largest organ of the human body and protects, not only the host against germ invasion, but also reflects the status of the whole body. The changes of the skin are easy to spot. To test validity of the constructed complexes, groups of volunteers were selected for testing the validity of the hypothesis. Two types of criteria subjective and objective were selected for these purposes. First, products were tested for toxicity (1). The question whether the antioxidant complexes present in the Biovien® products are biologically active and how well the products are adapted to needs of the individual at different stages of the life. In the next report, the volunteers validated the hypothesis that simultaneous application of Biovien® products, on top of the skin and per-orally is not only, not toxic, but also has an amplification effect (3). Summarized information is listed in this report indicating, that by using objective methods, it was possible to demonstrate normalization of the skin metabolic processes occurring after the application of Biovien®. Application of Biovien® products leads to improved defense of the human body. During volunteers testing, protocols developed at the School of Cosmetology Medical University of Lodz were used. During the last studies data was also collected from a Beauty Skin Master. Collected information leads to the conclusion, that Biovien® products are essential for improving functions of the innate defense system and maintenance of the barrier protecting the host against invading germs. Those finding open the new possibility for manipulation of not only skin chronic inflammatory disorders, but also others disorders that induce adverse reactions and shorten the life spun.

Material and Methods. A group of volunteers consisting of 128 individuals was selected among people of different age, with oily skin and acne problems, and other skin conditions. Many of them were previously treated by dermatologist and cosmetitien without success. The skin changes were evaluated and recorded according to the cosmetic criteria (protocol), developed in School of Cosmetology Medical University of Lodz, Poland. In addition, every volunteer passed through skin evaluation procedures where the skin pH, moisture, and skin lubrication status were measured (1,2,3) before initiation of the studies.

The objective variables were tested by means of the Beauty Skin Master imported from Italy. The skin pH, moisture, and sebum production on the surface of the skin was measured. Before initiation the basic testing, pilot studies were performed to asses the effect of the Biovien® cream and body lotion. In this evaluation study, carried out during three weeks, three groups of 15 volunteers each, were tested to asses whether the differences between Biovien® 4, 5, 6 cream and body lotion existed. The volunteers were tested for pH changes in weekly intervals. At the same time hydration level of the skin and production of the lubricant was determined. During this time of testing, decrease of pH of the skin was observed after the cream and body lotion was applied. The hydration level of the skin increased circa 3-4%, and level of lubrication increased 5-6%. The pH level dropped from a pre testing average pH 7.8 to pH 5.0. Since the skin surface of the face represent 10% of the whole body surface, the decision was made to test changes on the whole skin. The main study was carried out using Biovien® 1, 2, 4, 5, 6 body lotion. Since body lotion carries identical complexes, as the corresponding creams, testing the whole skin provides more accurate evaluation effects. Volunteers received body lotion to cover the surface of the skin two times a day for a period of ten weeks. The skin changes measured by Beauty Skin Master were performed, as in pilot study, in a weekly interval. Different variants were selected for analysis and evaluation of statistical significance.

Results. The Age distribution, skin changes, and remaining subjective variables were summarized in tables 24, 25, 26, 27, 28. The pre clinical evaluations provide similar results to those reported in the Examples 1, 2 and 3 above.

TABLE 24
The age distribution in different volunteers subgroups:

| Group: | | No | 14–20 | 21–25 | 26–30 | 31–35 | 36–40 | 41–45 | 46–50 | 51–55 | 56–65 |
|---|---|---|---|---|---|---|---|---|---|---|---|
| 7. Balsam | B1 | 14 | 4 | 9 | 1 | 0 | 0 | 0 | 0 | 0 | 0 |
| 8. Balsam | B2 | 15 | 5 | 10 | 0 | 0 | 0 | 0 | 0 | 0 | 0 |
| 9. Balsam | B4 | 20 | 0 | 0 | 0 | 0 | 9 | 4 | 4 | 3 | 0 |
| 10 Balsam | B5 | 20 | 0 | 0 | 0 | 0 | 0 | 8 | 5 | 4 | 3 |
| 11 Balsam | B6 | 14 | 0 | 0 | 0 | 0 | 0 | 3 | 4 | 5 | 2 |
| Total: | | 83 | 9 | 19 | 1 | 0 | 9 | 15 | 13 | 12 | 5 |

TABLE 25
The average, range, and difference in age are listed below.

| Group: | Average age: | Range | Difference |
|---|---|---|---|
| 7. | 21.5 | 19–24 | 5 |
| 8. | 20.3 | 19–24 | 5 |
| 9. | 480 | 41–58 | 17 |
| 10. | 48.0 | 41–58 | 17 |
| 11. | 50.1 | 42–57 | 15 |

TABLE 26
Further characterization of the groups assigned to different creams.
Values expressed in %:

| | Group: | | | | | |
|---|---|---|---|---|---|---|
| | B ®-1 | B ®-2 | B ®-3 | B ®-4 | B ®-5 | B ®-6 |
| Generalized | 16.7 | 20.0 | 0 | 0 | 0 | 0 |
| lesions; | | | | | | |
| Localized | 83.3 | 80.0 | 100.0 | 100.0 | 100.0 | 100.0 |
| lesions | | | | | | |
| Previous | 23.3 | 56.0 | 56.0 | 68.0 | 87.0 | 80.0 |
| treatment: | | | | | | |

TABLE 27
Status of the skin before the Biovien ® products application:
the frequency was expressed in %.

| | The group tested: | | | | | |
|---|---|---|---|---|---|---|
| | B-1 | B-2 | B-3 | B-4 | B-5 | B-6 |
| A. The type e skin: | | | | | | |
| Mixed | 20.0 | 66.7 | 26.6 | 44.0 | 40.9 | 50.0 |
| Dry | 3.3 | 0 | 0 | 56.0 | 59.1 | 50.0 |
| Oily | 73.3 | 33.3 | 71.4 | 0 | 0 | 0 |
| B. Carnation of the skin: | | | | | | |
| Bright to yellow | 20.0 | 51.3 | 71.4 | 80.9 | 63.6 | 70.0 |
| Gray | 53.3 | 41.0 | 8.0 | 0.0 | 4.5 | 0 |
| Dark | 6.7 | 7.7 | 9.5 | 20.0 | 31.8 | 35.0 |
| C. Vessels: | | | | | | |
| Singular | 26.7 | 7.7 | 42.9 | 32.0 | 31.8 | 32.5 |
| Numerous | 6.3 | 7.7 | 38.1 | 56.0 | 68.2 | 65.0 |
| Absent | 70.0 | 84.6 | 19.0 | 12.0 | 0 | 2.0 |
| D. Wrinkles: | | | | | | |
| Superficial | 6.7 | 33.3 | 66.7 | 24.0 | 3.0 | 25.0 |
| Numerous | 0.0 | 0.0 | 0.0 | 20.0 | 4.0 | 30.0 |
| Deep | 0.0 | 0.0 | 28.5 | 52.0 | 21.0 | 45.0 |
| Absent | 93.3 | 66.7 | 4.8 | 4.0 | 2.0 | 0.0 |
| E. Elasticity: | | | | | | |
| Normal | 100.0 | 100.0 | 4.8 | 8.0 | 9.0 | 5.0 |
| Slightly decreased | 0 | 0 | 33.2 | 14.3 | 18.2 | 17.5 |
| Decreased | 0 | 0 | 62.0 | 77.7 | 72.8 | 77.5 |

Information presented in table 27 (above) indicates that skin changes differ among young and older groups. The most frequently changes were: the skin type, wrinkles, elasticity of the skin and vessels formation. The lesions and other changes observed on the skin are listed in next table.

TABLE 28
The volunteers skin status before the Biovien ® cream application (%):

| Biovien Cream: | B ®-1 | B ®-2 | B ®-3 | B ®-4 | B ®-5 | B ®-6 |
|---|---|---|---|---|---|---|
| a. Acne | 10.0 | 2.6 | 4.8 | 8.0 | 0.0 | 2.5 |
| b. Skin discoloration | 20.0 | 43.6 | 38.1 | 52.0 | 100.0 | 96.5 |
| c. Comedo presence | 100.0 | 64.1 | 0 | 0 | 0 | 0 |
| d. Foci of inflammation | 100.0 | 76.4 | 0 | 0 | 0 | 0 |
| e. Hyper-keratinosis | 50.0 | 7.7 | 0 | 0 | 4.5 | 2.5 |
| g. Enlarged sebum vessels | 100.0 | 72.0 | 0 | 0 | 0 | 0 |
| h. Pores enlargement | 40.0 | 69.2 | 4.8 | 0 | 0 | 0 |
| i. Scars | 43.3 | 41.0 | 0 | 0 | 0 | 0 |
| j. Piling centers | 20.0 | 7.7 | 14.3 | 8 | 0 | 2.5 |

Changes seen on the skin of volunteers presented in table 28, indicates that Acne was distributed evenly among different groups. This may indicate that disease is of different etiology. Signs of the Oily skin disorder appeared only in B®-1 and B®-2 groups. In B®-3 group were seen only two variables; skin discoloration and enlargement of the skin pores.

The skin discoloration increases with the age of participants to the level where almost everybody was affected. The sebum over production occurs only among young participants.

TABLE 29
Initial pH changes seen on volunteers skin.

| | Body lotion no: | | | | | |
|---|---|---|---|---|---|---|
| | B ®1 | B ®-2 | B ®-4 | B ®-5 | B ®-6 | |
| Average pH | 6.64 | 6.49 | 7.03 | 6.98 | 7.28 | +0.64 |
| values | | | | | | |
| Range of pH | 6.5–6.9 | 6.2–6.7 | 6.3–7.9 | 6.3–7.9 | 6.9–7.9 | |
| Difference | 0.40 | 0.5 | 1.2 | 1.2 | 1.0 | |
| between pH | | | | | | |

The hydration effect on volunteers skin.

| | | | | | | |
|---|---|---|---|---|---|---|
| Hydration level % | 71.71 | 63.65 | 67.05 | 64.10 | 65.93 | −5.78 |
| Range | 74–67 | 70–74 | 75–60 | 73–60 | 72–61 | |
| Range of | 7.0 | 4.0 | 15.0 | 13.0 | 11.0 | |
| difference in % | | | | | | |

The sebum production among volunteers.

| | | | | | | |
|---|---|---|---|---|---|---|
| Sebum | 67.93 | 64.93 | 67.85 | 67.37 | 61.86 | −6.07 |
| production | | | | | | |
| Range | 69–65 | 70–68 | 76–60 | 76–60 | 79–60 | |
| Difference % | 4.0 | 2.0 | 16.0 | 16.0 | 19.0 | |

Presented data supports the observation that the young group is different from remaining. Data support thesis, that two different mechanisms of changes were observed. Information presented in table 6, shows the pH value, hydration, and sebum production slightly varied.

TABLE 30
pH changes of the skin during 10 weeks application Biovien ®-2.

| | | | Weeks: | | | | | | | | |
|---|---|---|---|---|---|---|---|---|---|---|---|
| No. | Age | Starting | 1 | 2 | 3 | 4 | 5 | 6 | 7 | 8 | 9 |
| 1. | 21 | 6.5 | 5.7 | 5.5 | 5.5 | 5.5 | 5.5 | 5.4 | 5.5 | 5.4 | 5.4 |
| 2. | 23 | 6.6 | 5.9 | 5.5 | 5.5 | 5.5 | 5.5 | 5.5 | 5.5 | 5.5 | 5.5 |
| 3. | 24 | 6.6 | 5.9 | 5.5 | 5.5 | 5.5 | 5.5 | 5.6 | 5.5 | 5.5 | 5.5 |
| 4. | 23 | 6.6 | 5.7 | 5.5 | 5.3 | 5.4 | 5.4 | 5.5 | 5.4 | 5.4 | 5.4 |
| 5. | 20 | 6.7 | 6.1 | 5.8 | 5.6 | 5.6 | 5.5 | 5.6 | 5.6 | 5.5 | 5.5 |
| 6. | 19 | 6.4 | 5.9 | 5.7 | 5.7 | 5.5 | 5.6 | 5.6 | 5.6 | 5.6 | 5.6 |
| 7. | 22 | 6.5 | 5.9 | 5.5 | 5.5 | 5.4 | 5.5 | 5.4 | 5.6 | 5.5 | 5.6 |
| 8. | 19 | 6.3 | 5.7 | 5.5 | 5.5 | 5.5 | 5.5 | 5.6 | 5.6 | 5.5 | 5.6 |
| 9. | 19 | 6.6 | 5.8 | 5.5 | 5.5 | 5.5 | 5.5 | 5.6 | 5.5 | 5.5 | 5.5 |
| 10. | 21 | 6.2 | 5.7 | 5.5 | 5.5 | 5.4 | 5.5 | 5.5 | 5.4 | 5.5 | 5.5 |
| 11. | 24 | 6.6 | 5.9 | 5.5 | 5.5 | 5.6 | 5.6 | 5.5 | 5.5 | 5.5 | 5.5 |
| 12. | 24 | 6.7 | 5.7 | 5.5 | 5.5 | 5.6 | 5.5 | 5.6 | 5.5 | 5.5 | 5.6 |
| 13. | 22 | 6.7 | 5.9 | 5.5 | 5.5 | 5.5 | 5.5 | 5.6 | 5.5 | 5.5 | 5.6 |
| 14. | 20 | 6.6 | 6.0 | 5.7 | 5.5 | 5.4 | 5.5 | 5.5 | 5.5 | 5.4 | 5.5 |
| 15. | 23 | 6.7 | 6.0 | 5.5 | 5.5 | 5.5 | 5.6 | 5.6 | 5.6 | 5.6 | 5.6 |

| | | | | | | | | | | |
|---|---|---|---|---|---|---|---|---|---|---|
| Mean: | 6.533 | 5.8533 | 5.5464 | 5.5067 | 5.4933 | 5.5133 | 5.4000 | 5.5200 | 5.4933 | 5.5267 |
| SD | 0.1506 | 0.1302 | 0.0990 | 0.0799 | 0.0704 | 0.0516 | 0.0737 | 0.0676 | 0.0594 | 0.0704 |
| SE Mean | 0.0389 | 0.0336 | 0.0256 | 0.0206 | 0.0182 | 0.0133 | 0.0190 | 0.0175 | 0.0153 | 0.0182 |
| Minimum | 6.2000 | 5.7000 | 5.5000 | 5.3000 | 5.4000 | 5.4000 | 5.4000 | 5.4000 | 5.4000 | 5.4000 |
| Maximum | 6.7000 | 6.1000 | 5.5000 | 5.7000 | 5.6000 | 6.6000 | 6.6000 | 5.6000 | 5.6000 | 5.6000 |

### Analysis of variants, changes of the skin pH of volunteers using the Biovien® 2 body lotion.

| Source | DF | SS | MS | F | P |
|---|---|---|---|---|---|
| Weak (A) | 9 | 15.0051 | 1.66723 | 203.56 | 0.0000 |
| Residual | 140 | 1.14667 | 0.00819 | | |
| Total | 149 | 16.1517 | | | |

### Comments: there are 3 groups in which the means are not significantly different from one another.

| | |
|---|---|
| Critical T Value | 1.977 |
| Critical value for comparison | 0.0653 |
| Standard error for comparison | 0.0330 |
| Error term used: Residual, | 140 DF. |
| Rejection level | 0.050 |

### P value is out of scale because of F value.

TABLE 31
pH changes of the skin during 10 weeks application Biovien ®-5.

| | | | Weeks: | | | | | | | | | |
|---|---|---|---|---|---|---|---|---|---|---|---|---|
| No. | Age | Starting | 1 | 2 | 3 | 4 | 5 | 6 | 7 | 8 | 9 | 10 |
| 1. | 45 | 7.3 | 6.3 | 6.2 | 5.7 | 5.6 | 5.8 | 5.4 | 5.5 | 5.2 | 5.3 | 5.3 |
| 2. | 47 | 7.3 | 5.6 | 5.6 | 5.7 | 5.6 | 5.5 | 5.4 | 5.5 | 5.3 | 5.5 | 5.3 |
| 3 | 48 | 7.1 | 6.5 | 5.9 | 5.7 | 5.6 | 5.5 | 5.4 | 5.5 | 5.6 | 5.4 | 5.5 |
| 4. | 42 | 7.8 | 6.3 | 6.2 | 5.7 | 5.6 | 5.7 | 5.6 | 5.7 | 5.4 | 5.5 | 5.6 |
| 5. | 55 | 6.3 | 6.1 | 5.8 | 5.4 | 5.5 | 5.4 | 5.5 | 5.6 | 5.4 | 5.4 | 5.6 |
| 6. | 54 | 7.0 | 5.9 | 5.9 | 5.7 | 5.6 | 5.5 | 5.6 | 5.7 | 5.5 | 5.7 | 5.6 |
| 7. | 43 | 7.9 | 6.3 | 5.9 | 6.0 | 5.6 | 5.8 | 5.4 | 5.5 | 5.7 | 5.5 | 5.3 |
| 8. | 47 | 7.8 | 6.3 | 5.2 | 5.7 | 5.6 | 5.8 | 5.4 | 5.5 | 5.2 | 5.3 | 5.3 |
| 9. | 55 | 7.0 | 6.3 | 6.0 | 5.7 | 5.6 | 5.5 | 5.4 | 5.5 | 5.6 | 5.3 | 5.5 |
| 10. | 58 | 7.2 | 6.8 | 5.9 | 5.7 | 5.8 | 5.7 | 5.8 | 5.8 | 5.8 | 5.8 | 5.8 |
| 11. | 45 | 7.8 | 7.3 | 6.2 | 5.7 | 5.6 | 5.8 | 5.4 | 5.5 | 5.4 | 5.3 | — |
| 12. | 58 | 7.3 | 6.8 | 6.2 | 5.7 | 5.6 | 5.8 | 5.8 | 5.6 | 5.7 | 5.7 | 5.7 |
| 13. | 45 | 7.3 | 6.3 | 5.9 | 5.9 | 5.6 | 5.8 | 5.6 | 5.7 | 5.7 | 5.7 | 5.7 |
| 14 | 45 | 7.3 | 6.5 | 5.9 | 5.8 | 5.7 | 5.6 | 5.4 | 5.5 | 5.6 | 5.5 | 5.6 |
| 15. | 53 | 7.1 | 6.8 | 6.8 | 6.7 | 6.5 | 5.8 | 5.7 | 5.7 | 5.8 | 5.7 | 5.6 |
| 16. | 42 | 7.3 | 6.3 | 6.3 | 5.7 | 5.6 | 5.8 | 5.6 | 5.7 | 5.7 | 5.7 | 5.7 |
| 17. | 47 | 7.3 | 6.7 | 6.2 | 5.7 | 5.6 | 5.8 | 5.7 | 5.5 | 5.8 | 5.7 | 5.3 |
| 18 | 41 | 7.3 | 6.3 | 6.2 | 5.7 | 5.6 | 5.8 | 5.6 | 5.5 | 5.8 | 5.7 | 5.7 |
| 19 | 44 | 7.2 | 6.5 | 5.7 | 5.7 | 5.6 | 5.8 | 5.7 | 5.8 | 5.7 | 5.7 | 5.7 |
| 20 | 43 | 7.3 | 6.5 | 5.9 | 5.7 | 5.6 | 5.7 | 5.6 | 5.6 | 5.6 | 5.6 | 5.7 |

| | | | | | | | | | | | |
|---|---|---|---|---|---|---|---|---|---|---|---|
| Mean | 7.2950 | 6.4200 | 5.9950 | 5.7650 | 5.6550 | 5.6950 | 5.5500 | 5.5950 | 5.5750 | 5.5500 | 5.5526 |
| SD | 0.3546 | 0.3607 | 0.3220 | 0.2455 | 0.2064 | 0.1395 | 0.1433 | 0.1099 | 0.1970 | 0.0380 | 0.1712 |
| SE Mean | 0.0793 | 0.0807 | 0.0720 | 0.0549 | 0.0462 | 0.0312 | 0.0320 | 0.0246 | 0.0441 | 0.0380 | 0.0393 |
| Minimum | 6.3000 | 5.6000 | 5.2000 | 5.4000 | 5.5000 | 5.4000 | 5.4000 | 5.5000 | 5.2000 | 5.3000 | 5.3000 |
| Maximum | 7.9000 | 7.3000 | 6.8000 | 6.7000 | 6.5000 | 5.8000 | 5.8000 | 5.8000 | 5.8000 | 5.8000 | 5.8000 |

### Analysis of variants, changes of the skin pH of volunteers using the Biovien® 4 body lotion.

| Source | DF | SS | MS | F | P |
|---|---|---|---|---|---|
| Weak (A) | 10 | 57.9793 | 5.79793 | 104.05 | 0.0000 |
| Residual | 208 | 11.5899 | 0.05572 | 0.05572 | |
| Total | 218 | 69.5691 | | | |

### Comments: There are 5 groups in which the means are not significantly different from one another.

| | |
|---|---|
| Critical T Value | 1.971 |
| Critical value for comparison | 0.1472 |
| Standard error for comparison | 0.0746 |
| Error term used: Residual, | 208 DF. |
| Rejection level | 0.050 |

### P value is out of scale because of F value.

TABLE 32
pH changes of the skin during 10 weeks application Biovien ®-5.

| | | | Weeks: | | | | | | | | | |
|---|---|---|---|---|---|---|---|---|---|---|---|---|
| No. | Age | Starting | 1 | 2 | 3 | 4 | 5 | 6 | 7 | 8 | 9 | 10 |
| 1. | 47 | 7.3 | 6.5 | 5.8 | 5.7 | 5.6 | 5.7 | 5.7 | 5.7 | 5.7 | 5.7 | 5.6 |
| 2. | 47 | 7.3 | 5.6 | 5.6 | 5.7 | 5.6 | 5.7 | 5.7 | 5.8 | 5.3 | 5.5 | 5.3 |
| 3. | 48 | 7.1 | 6.5 | 5.9 | 5.7 | 5.6 | 5.5 | 5.4 | 5.5 | 5.6 | 5.4 | 5.5 |
| 4. | 42 | 7.8 | 6.3 | 6.2 | 5.7 | 5.6 | 5.7 | 5.6 | 5.7 | 5.4 | 5.5 | 5.6 |
| 5. | 55 | 6.3 | 6.1 | 5.8 | 5.4 | 5.5 | 5.4 | 5.5 | 5.6 | 5.4 | 5.4 | 5.6 |
| 6. | 54 | 7.0 | 5.9 | 5.9 | 5.7 | 5.6 | 5.5 | 5.6 | 5.7 | 5.5 | 5.7 | 5.6 |
| 7. | 43 | 7.9 | 6.3 | 5.9 | 6.0 | 5.6 | 5.8 | 5.4 | 5.5 | 5.7 | 5.5 | 5.3 |
| 8. | 47 | 7.8 | 6.3 | 5.2 | 5.7 | 5.6 | 5.8 | 5.4 | 5.5 | 5.2 | 5.3 | 5.3 |
| 9. | 55 | 7.0 | 6.3 | 6.0 | 5.7 | 5.8 | 5.7 | 5.8 | 5.8 | 5.8 | 5.8 | 5.8 |
| 11. | 45 | 7.8 | 7.3 | 6.2 | 5.7 | 5.6 | 5.8 | 5.4 | 5.5 | 5.4 | 5.3 | — |
| 12. | 58 | 7.3 | 6.8 | 6.2 | 5.7 | 5.6 | 5.8 | 5.8 | 5.6 | 5.8 | 5.8 | 5.7 |
| 13. | 45 | 7.3 | 6.3 | 5.9 | 5.9 | 5.6 | 5.8 | 5.6 | 5.7 | 5.7 | 5.7 | 5.7 |
| 14. | 45 | 7.3 | 6.5 | 5.9 | 5.8 | 5.7 | 5.6 | 5.4 | 5.5 | 5.6 | 5.5 | 5.6 |
| 15. | 53 | 7.1 | 6.8 | 6.8 | 6.7 | 6.5 | 5.8 | 5.7 | 5.7 | 5.8 | 5.7 | 5.6 |
| 16. | 42 | 7.3 | 6.3 | 6.3 | 5.7 | 5.6 | 5.8 | 5.6 | 5.7 | 5.7 | 5.7 | 5.7 |
| 17. | 47 | 7.3 | 6.7 | 6.2 | 5.7 | 5.6 | 5.8 | 5.7 | 5.5 | 5.8 | 5.7 | 5.3 |
| 18. | 41 | 7.3 | 6.3 | 6.2 | 5.7 | 5.6 | 5.8 | 5.6 | 5.5 | 5.8 | 5.7 | 5.7 |
| 19. | 44 | 7.2 | 6.5 | 5.7 | 5.7 | 5.6 | 5.8 | 5.7 | 5.8 | 5.7 | 5.7 | 5.7 |
| 20. | 43 | 7.8 | 7.3 | 6.7 | 5.9 | 5.7 | 5.6 | 5.7 | 5.6 | 5.6 | 5.6 | 5.7 |

| | | | | | | | | | | | |
|---|---|---|---|---|---|---|---|---|---|---|---|
| Mean | 7.3200 | 6.4700 | 6.0050 | 5.7750 | 5.6600 | 5.6950 | 5.5850 | 5.6200 | 5.6050 | 5.5750 | 5.2900 |
| SD | 0.3722 | 0.4092 | 0.3410 | 0.2468 | 0.2062 | 0.1317 | 0.1424 | 0.1152 | 01820 | 0.1682 | 0.2548 |
| SE Mean | 0.0832 | 0.0915 | 0.0763 | 0.0552 | 0.0461 | 0.0294 | 0.0319 | 0.0258 | 0.0407 | 0.0376 | 0.2806 |
| Minimum | 6.3000 | 5.6000 | 5.2000 | 5.4000 | 5.5000 | 5.4000 | 5.4000 | 5.5000 | 5.2000 | 5.3000 | 0.0000 |
| Maximum | 7.9000 | 7.3000 | 6.8000 | 6.7000 | 6.5000 | 5.8000 | 5.4000 | 5.8000 | 5.8000 | 5.8000 | 5.8000 |

### Analysis of variants, changes of the skin pH of volunteers using the Biovien® 4 body lotion.

| Source | DF | SS | MS | F | P |
|---|---|---|---|---|---|
| Weak (A) | 10 | 64.0354 | 6.40354 | 31.83 | 0.0000 |
| Residual | 209 | 42.0410 | 0.20115 | | |
| Total | 219 | 106.076 | | | |

### Comments: There are 5 groups in which the means are not significantly different from one another.

| | |
|---|---|
| Critical T Value | 1.971 |
| Critical value for comparison | 0.2796 |
| Standard error for comparison | 0.1418 |
| Error term used: Residual, | 209 DF. |
| Rejection level | 0.050 |

### P value is out of scale because of F value.

TABLE 33
pH changes of the skin during 10 weeks application Biovien ®-6.

| | | | Weeks: | | | | | | | | | |
|---|---|---|---|---|---|---|---|---|---|---|---|---|
| No | Age | Starting | 1 | 2 | 3 | 4 | 5 | 6 | 7 | 8 | 9 | 10 |
| 1 | 55 | 7.5 | 5.8 | 5.5 | 5.3 | 5.5 | 5.4 | 5.4 | 5.5 | 5.6 | 5.6 | 5.6 |
| 2 | 57 | 7.7 | 6.6 | 5.6 | 5.7 | 5.6 | 5.5 | 5.5 | 5.5 | 5.7 | 5.6 | 5.6 |
| 3. | 58 | 7.9 | 6.7 | 5.9 | 5.7 | 5.6 | 5.5 | 5.5 | 5.5 | 5.6 | 5.7 | 5.7 |
| 4. | 52 | 7.8 | 6.7 | 6.2 | 5.7 | 5.6 | 5.7 | 5.6 | 5.7 | 5.7 | 5.7 | 5.6 |
| 5. | 52 | 6.9 | 6.1 | 5.8 | 5.7 | 5.7 | 5.7 | 5.7 | 5.7 | 5.7 | 5.7 | 5.7 |
| 6. | 42 | 7.0 | 6.5 | 5.9 | 5.7 | 5.6 | 5.6 | 6.6 | 5.7 | 5.7 | 5.7 | 5.5 |
| 7. | 43 | 7.2 | 6.5 | 5.9 | 6.0 | 5.6 | 5.7 | 5.7 | 5.7 | 5.7 | 5.7 | 5.6 |
| 8. | 48 | 7.1 | 6.3 | 5.7 | 5.7 | 5.7 | 5.6 | 5.6 | 5.6 | 5.7 | 5.8 | 5.7 |
| 9. | 53 | 7.0 | 6.7 | 6.0 | 5.7 | 5.8 | 5.8 | 5.8 | 5.7 | 5.6 | 5.7 | 5.7 |
| 10. | 52 | 7.2 | 6.7 | 5.9 | 5.7 | 5.7 | 5.8 | 5.7 | 5.7 | 5.7 | 5.7 | 5.8 |
| 11. | 47 | 7.2 | 7.0 | 6.5 | 5.7 | 5.7 | 5.8 | 5.7 | 5.7 | 5.7 | 5.7 | — |
| 12. | 52 | 7.7 | 6.8 | 6.1 | 5.8 | 5.8 | 5.8 | 5.7 | 5.7 | 5.7 | 5.7 | 5.7 |
| 13. | 45 | 7.0 | 6.3 | 5.8 | 5.8 | 5.7 | 5.8 | 5.7 | 5.7 | 5.7 | 5.7 | 5.7 |
| 14. | 46 | 7.3 | 6.7 | 5.9 | 5.8 | 5.7 | 5.7 | 5.5 | 5.5 | 5.7 | 5.8 | 5.7 |

| | | | | | | | | | | | |
|---|---|---|---|---|---|---|---|---|---|---|---|
| Mean | 7.3214 | 6.5286 | 5.9071 | 5.7143 | 5.6643 | 5.6714 | 5.6929 | 5.6357 | 5.6714 | 5.7000 | 5.6615 |
| SD | 0.3355 | 0.3124 | 0.2495 | 0.1460 | 0.0842 | 0.1326 | 0.2841 | 0.0929 | 0.0469 | 0.0555 | 0.0768 |
| SE Mean | 0.0897 | 0.0835 | 0.0667 | 0.0390 | 0.0225 | 0.0354 | 0.0759 | 0.0248 | 0.0469 | 0.0148 | 0.0213 |
| Minimum | 6.9000 | 5.8000 | 5.5000 | 5.3000 | 5.5000 | 5.4000 | 5.4000 | 5.5000 | 5.6000 | 5.6000 | 5.5000 |
| Maximum | 7.9000 | 7.0000 | 6.5000 | 6.0000 | 5.8000 | 5.8000 | 6.6000 | 5.7000 | 5.7000 | 5.8000 | 5.8000 |

### Analysis of variants, changes of the skin pH of volunteers using the Biovien® 4 body lotion.

| Source | DF | SS | MS | F | P |
|---|---|---|---|---|---|
| Weak (A) | 10 | 39.3154 | 3.93154 | 102.62 | 0.0000 |
| Residual | 142 | 5.44005 | 0.03831 | | |
| Total | 152 | 44.7554 | | | |

### Comments: There are 4 groups in which the means are not significantly different from one another.

| | |
|---|---|
| Critical T Value | 1.977 |
| Critical value for comparison | 0.1462 |
| Standard error for comparison | 0.0740 |
| Error term used: Residual,. | 142 DF |
| Rejection level | 0.050 |

For example changes of pH of the skin using Biovien® 1 body lotion looks like is shown below. P value is out of scale because of F value.

Figure shows the changes in pH of the volunteer's skin treated with Biovien® 1 body lotion. Please note that it takes 4 weeks of daily application of Biovien®-1 body lotion to return the skin pH to normal value and stabilized pH 5.5 level. Thereafter only small variation were noticed. The kinetic of stabilization of the pH, differ for each product. Compare tables 30-33. This indicates that designed antioxidant complexes are different in their strength. To reach the goal of stabilization of the skin pH, Biovien®-1 body lotion required 4 weeks, Biovien®-2 body lotion needs 6 weeks, Biovien®-4, 5 weeks, Biovien®-5 6 weeks and Biovien®-6 only 4 weeks. It is worth to note that remaining two variables, the moisturizing skin and sebum production, responded to Biovien® products differently. Moisture of the skin among volunteers from group 1 and 2 improved marginally in comparison with groups 4, 5, 6. See Table 32. Similar pattern was observed in modulation of the lubricant production. The increase of the sebum production in group 1 and 2 was smaller than in group of 4, 5, 6. Presented results are in good agreements with logical prediction. Volunteers in group B®-4, B®-5 and B®-6 require more lubricant for their skin to enrich physiological conditions than group B®-1 and B®-2.

Results. A total of 443 volunteers participated in these studies. They used on their skin, Biovien® cream 1-6 or Biovien® Body lotion alone, or cream in combination with diet supplement. The time of administration was different. In pilot studies, the time of skin exposure to Biovien® was short (three weeks) whereas during basic exposure to product was extended to 10 weeks. During all those cycles of testing, none of volunteers reported signs of any adverse reaction like: aching, tingling, itching swelling or reddening. These signs represent the classical elements of inflammation. Inflammation responses, sometimes called “irritation symptoms” are indicators of the free radicals over production. In addition none of the 443 volunteers reported any sign affecting existing allergies (or sign of free radicals activation). In contrast all volunteers underlined beneficial effect on the skin as well as effect and well being feeling. The subjective signs of skin improvement after Biovien® body lotion application were similar to those in Examples 1, 2 and 3. The dramatic effect on skin pH was unexpected. Statistical evaluation showed extremely high P values that in analysis of variance went out of scale. Also skin moisture was improved and the same holds for sebum production. The younger groups increased sebum production in not statistically significant manner, however the changes in group that had dry skin increased significantly.

In every day situation the free radicals are generated by different enzymes or factors accompanying the germ invasion, germ's toxins or environmental impact. The level of free radicals production differs, depending upon the intensity of the impact. In certain situation the raise will be so weak and will not be noticed. In others, the free radicals may reach a level that may initiate signs of acute disease. Such condition may last couple days or weeks until the host defenses, gain control over the negative impact, and free radicals return to normal level. This negative impact of different etiology may lead to metabolic shock that frequently leads to the host death. In another scenarios the impact is strong, however the host defense overcomes it, but the immune system fails to eliminate the invading agent. The level of free radicals subsides. 4 Hydroxy-noneal (4HNE) compound that carries free radical character, but in addition can initiate process of apoptosis (4). The apoptosis reflects the severity of the impact. If the programmed cell death process (apoptosis) prevents repair forces, the host enters in to a condition called chronic diseases. The last one can be of degenerative or infectious nature. Both are shortening the host life. Acne with oily skin or Alzheimer's disease may serve as examples. To this category belong also: Rheumatoid arthritis, Arteriosclerosis, Diabetes type II and Overweight. All of these diseases are characterized by active apoptosis process and elevated level of free radicals that sustain production of 4-HNE and in turn the apoptosis process. Both chronic degenerative and infectious disorders may be considered as self perpetuating oxidative stress diseases.

The second class of disorders could be considered as the result of negative impacts of various infectious agents like: Chronic Hepatitis B, C, HIV, and Tuberculosis, Malaria and many others. The free radicals in those cases are induced by germs that block host metabolism at a specific level, for directing host energy, for growth of invading microorganism. When germs are not eliminated from the body during an acute phase of infection, the host activates a reaction or the law called tachophylaxis. This reaction inhibits human responses to invading germs and creates a condition for germs to remain inside the host for prolong period of time. For example during Acne or Hepatitis B infections victims can't induce antibodies that could eliminate germs or virus. The damages induced to host, generate elevated levels of the free radicals. The situation may reach dramatic proportion, when the host develops two chronic diseases simultaneously; degenerative and infectious or two types of infectious disorders. In this situation the host may loose control over production of free radicals and they may induce such quantities of free radicals that they reach shock level leading to premature host death. Application of modern drugs, like enzymes inhibitors (that by themselves are free radicals), induces similar production of free radicals as do for example viruses. Such situations may worsen the condition leading to patients death. Philosophy supporting the use of maximal tolerating doses of those inhibitors frequently leads to a tragic ending. Depending on dose of the inhibitor use, the free radicals production may increase exponentially or remain on an acceptable level. To avoid such type of reactions, the host introduced mechanism that amplify controlling compound's signals and overcome negative effects generated by free radicals. Such mechanism occurred when the same complex of scavengers was used topically and orally. Unfortunately, practitioner physician treating the patients may inadvertently be responsible for induction of oxidative stress, because of dominance of maximum tolerable doses philosophy. Among practicing colleagues circulate a saying that each one has its own private cemetery. Lack of a clear understanding as to what free radicals can do is the responsible element to blame. In such situations the host may enter the state called chronic disease. At this stage, free radicals induce production of the 4 HNE.

The beneficial effect of Biovien® products in younger groups with Acne, was presented in each of Examples 2, 3 and 4 present a new approach in controlling this disorder. The beneficial effect was demonstrated by disappearance of inflammation, acne, comedons, diminishing production of sebum, and return skin to physiological condition. These results occurred regularly after prolonged application of Biovien-1 or 2 cream. The incubation period between initiation of cream application and appearance of signs of healing, could be improved considerably by application of cream with the same biologically active elements delivered orally (See Example 3). Shortening the time necessary to effect appearance has an amplification character since responses were stronger than action of each component given separately. The syndrome does not return, providing the skin was treated sufficiently for a long enough period of time, to give the skin time to repair the metabolic damages induced during environmental impact. The oily skin disorder disappeared following Biovien® application and did not appear again. The witnesses are those volunteers who returned for follow up examination half year later. Presented data provides strong evidence that by changing the focus to the control of free radicals and the application of natural products properly mixed together, it was possible to repair damages and restore function of the largest organ of human body without induction of any side effects. This new method presented combating chronic oily skin is an alternative to today's anti acne therapy. Today's methods of Acne elimination are based on not acceptable microbiologist principles. Their methods involve procedures that are very hash and abrasive to the skin like cleansing, exfoliating and regenerating the skin. To control acne, all three steps employ compounds like: Salicylic acid, Alcohol, Benzoyl peroxide, Antibiotics, Alpha hydroxyl acid, selected oil, Resorcinol, Sulfur and Accutane. The character of these compounds may be demonstrated by the last compound on the list. Given indiscriminately to pregnant women, may cause birth defects in up to 90% of cases. All those compounds were used to kills germs during the cleansing and exfoliating steps. Both steps effectively eliminate the dead layer of the skin and the presence of germs. But germs at this stage of acne are present also in skin gland, producing lubricant for skin and in the deeper layer of skin, inside the true skin. The keratinocites need to divide several times. To repair the protector of the layer of dead cells, the reconstruction process requires time to accomplish that task. If cells divide with the speed of one division every 24 hours, repairing the damage would take several days. In mean time bacteria that survive this harsh treatment start to divide every 5 minutes. During the time of restoration, the barrier germs population returns to normal level (60 minutes divide by 5 gives you the number of bacterial generation within 1 hour of time×24 gives you the number of germ division during the day. 60:5×24=288 generation or 82944 germs from initial one germ). From studies on the elimination of germs from skin, we know that even 20-30 minutes washing of the surgeon hands with soap and water did not eliminate the germs from the skin. The view the above takes in an attempt to eliminate the skin germs look silly, in addition to the application of abrasive, toxic free radicals producing treatment. The whole procedure does not make sense. The results of such procedures were seen among numbers of volunteers who failed to eliminate acne during previous treatment before enrollment to this study Further, consequences of Biovien® application open the possibility to extend a protective “umbrella” on the skin at different stages of the human life. Observed shortening the time span between application and effect of Biovien® cream (incubation period) indicates that the designed new approach operates within physiological borders and repairs natural defenses. Protection of natural defenses leads towards the control of existing disorder.

This last conclusion is based upon the fact that Biovien® products modulate skin pH, humidity and sebum production, and signs of free radical damages. This has broad biological significance. It opens a totally new opportunity to control and eliminate not only chronic Acne inflammations, but also other chronic degenerative and infectious processes of adulthoods that in consequences shorten the human life spun. There is strong circumstantial evidence that the new method of application of antioxidant complexes present in Biovien® series, can act on other chronic diseases like: Rheumatoid arthritis, Arteriosclerosis, Diabetes type II or Obesity. All of those chronic progressive disorders are characterized as results of the free radicals unbalances that induce process of apoptosis. New procedures permit to design control of free radicals to such levels that they diminish the condition for appearance of chronic infectious diseases like: Tuberculosis, Chronic Viral Hepatitis type B, C, Immunodeficiency disease and others. Decreasing the level of the oxidative stress releases tachophylaxis and permits the immune system to build specific antibodies that eliminate Hepatitis B and C viruses. In addition will diminish appearance of liver cancer (Hepato-cellular carcinoma) connected with those viruses. Extending this approach further, it is possible to predict that several chronic human diseases can be controlled and even eradicated by the application of designed antioxidant complexes. HIV and Alzheimer's diseases should be considered as closest candidates for eradication attempts, since information exists to believe such procedures could be successful. The logical reasoning presented above found support in published laboratory studies (12) and confirmation from present clinical reports. Changes of the skin pH after cream or body lotion application indicate that effects of Biovien® anti free radical complexes can be measured by objective methods. Generated objective data indicates that Biovien® series not only provides maintenance of the skin, but also has a profound impact on the human defenses. The Biovien® products not only change the pH of the skin to its normal level, but also modulate the skin moisture and sebum production. Correction of the above variables indicates that anti free radicals management has an effect, not only on the surface of the skin, but also on functions of the immune system. Induction of the disappearance of acne, oily skin lesion, and returning the skin to normal condition, indicates that antioxidant complexes applied have a “cross the border” effect not yet observed. This finding encourages us to use similar reasoning to different situations. In order to be successful you need to have profound knowledge concerning free radicals and the mechanisms governing the immune system.

Recently published studies in the American Medical Association Journal support the present invention. In 68 different major studies involving 128,000 people evaluating vitamin A, C, E and beta carotene complexes, the authors of these studies irrevocably pointed out that selected scavengers are not effective. For example it is impossible to expect the curative effect of the vitamin C in cases of Beri-Beri disease that are caused by lack of vitamin B-1. In reverse the scurvy can not be cured by Vitamin B-1. Application of wrong assumptions in the 68 studies involving more than 128,000 people led to generate the conclusions that antioxidants may have a negative impact on health of human population. Those conclusion based on flawed methodologically studies, reached totally absurd results and conclusion. The authors of these studies did not report which of the anti oxidant cocktails components used by them act as inhibitors of free radicals production, which neutralizes existing free radicals, and which serve as chaperones controlling intracellular level of the free radicals. Certainly none of the authors took under consideration chaperones and inhibitors of free radicals as the elements that play an essential role in true free radicals management. Their presented observations are also in sharp contrast with existing reports that claim the development of allergy or skin irritations up to 5. % of tested participants after using authors cosmetics. The claims are in good agreement with the reports from our volunteers that they tested different cosmetics before signing in to our studies. Comparison of Biovien® product effects with effects reported by others is in sharp contrast. Biovien® products do not induce antibodies or allergies. Presented observation leads to the conclusion that the application of scavengers inhibiting free radicals formation, anti-oxy vitamins species, scavenging existing free radicals together with chaperones modulating free radicals inside the cell, will be effective. These properties in these complexes are far superior than the previously used compounds such as antibiotics and allergies.

The physiological changes associated with aging include impairment of the barrier, decreased turnover of epidermal cells, reduce numbers of keratinocytes and fibroblast and a reduced vascular network. Prevention of the skin senescence processes relies in part on protection the skin from sun, tobacco, and avoidance of sustained mechanical stress to the skin. The scavengers of free radicals, in adequate combinations are major contributors to slow down the aging process. Vitamins called by us (anti-oxi-vitamins), focused on free radicals, showed positive effects in protecting the skin against environmental insults. Since appearance of the skin depends not only upon the environment, but also on the metabolic and immune condition of the host, the inventors developed a strategy to deliver active compounds for simultaneous topical and oral delivery. These complexes exhibit powerful protection and regeneration activity.

Another element that needs to be pointed out is the role of oxide-reductive stress and its effect on human body metabolism. For example, majority of germs on the skin are dependent on iron. The lack of free iron ion causes death of those bacteria. By binding the iron by metalloprotein, Fenton's reaction is inhibited and therefore the growth of the iron dependent germ is inhibited. Lack of the free iron and blocking the flow of energy to germs generated by the iron oxidation process, kills the germs. The same holds true for other transitional metals like copper or Zinc. The free iron in addition upon oxidation generates free radicals called hydroxyl. They are directly responsible for skin damage, wrinkles formation and 4HNE formation. Elimination of iron ions from the skin not only prevent the formation changes mentioned above, but also keeps the skin young and healthy.

The success was achieved thanks to precise understanding of the mechanisms governing the oily skin disorder. The new procedure is particularly important for a young biologically active person. The biologically active compounds introduced in Biovien® series are not only powerful antibacterial agents that inhibits the growth of iron or metal dependent bacteria, but are also factors modulating the metabolism of the skin and the whole body, correcting for example, overproduction of the lubricant. As a result of these changes, production of the oily secretion is corrected, the skin appearance improved, even during menstruation period. As can be seen the Biovien® 1 and 2 creams and body lotion exhibits double action; act as germ growth inhibitor and limiting the free radicals formation inhibiting wrinkle formation. These two properties permit us to consider Biovien®-1 cream as an anti aging factor acting in the very early stages of human life. These unique properties place Biovien®-1, 2 creams, body lotions and capsules in a unique category of essential nutrients provider and not true cosmetics. Present evaluation studies provide proof that our theoretical predictions were correct. The study suggested also, that these products are effective not only for the skin, but also for the whole body.

### EXAMPLE 5

Additional formulations have been prepared for specific applications of the present invention, with specific ranges for use. The product line disclosed herein includes the control of free radicals, which are responsible for the chronic inflammatory, degenerative processes and aging of the human body. Most human chronic diseases of infectious and degenerative nature are the result of a dysfunction of the host defense system, caused by a disruption in the communications network, mainly due to Free Radicals. Prolonged application of Biovien products may be used to control free radical formation. The methods disclosed herein have been shown (Examples—above) to improvement of these conditions and their possible elimination.

Another benefit of controlling free radicals with our products is the protection of the human native defense system against viruses and other surrounding germs. Complexes were designed with biologically active substances called anti-oxy-vitamins and chaperones, adapted to the requirement of the applicants' age.

Composition of the Biovien® 1-7 creams.

| | Concentration range in | |
|---|---|---|
| Material | % of total mass | Preferred Range |
| 1. Tego ® care 450 | 7.0–0.1 | 4.0–2.0 |
| 2. Tego ® M | 7.0–0.1 | 4.0–2.0 |
| 3. Tego ® Alkohol 1618 | 7.0–0.1 | 2.0–0.5 |
| 4. Tegosoft ® OS | 10.0–0.3 | 7.0–3.0 |
| 5. Acylan LA 0478 | 7.0–1.0 | 3.0–1.0 |
| 6. Vitis venifera | 10.0–0.5 | 3.0–0.5 |
| 7. Soybean oil | 10.0–0.5 | 3.0–1.0 |
| 8. Abil 350 | 4.0–0.1 | 1.5–0.5 |
| 9. Paraffin oil | 5.0–0.5 | 3.0–0.5 |
| 10. C.L.A. | 3.0–0.1 | 1.0–0.3 |
| 11. Vit. E | 3.0–0.1 | 2.0–0.5 |
| 12. Lactoferrin | 7.0–0.1 | 2.0–0.5 |
| 13. Hippohae Ramn. | 10.0–0.1 | 2.0–0.5 |
| 14. Flax oil | 10.0–0.1 | 1.0–0.2 |
| 15. PRP-peptides | 0.001–0.000001 | 0.0001–0.00001 |
| 16. Anthocyanes | 1.0–0–0001. | 0.1–0.001 |
| 17. Glycerin | 10.0–0.1 | 4.0–0.5 |
| 18. Oxynex K liquid | 7.0–0.01 | 0.5–0.1 |
| 19. Geogard ® ultra | 7.0–0.5 | 3.0–0.8 |
| 20. Methyl-paraben | 3.0–0.1 | 3.0–0.1 |
| 21. Propyl-paraben | 3.0–0.1 | 2.0–1.0 |
| 22. 20% citric acid | 1.0–0.01 | 0.5–0.01 |
| 23. Water add to 100% | | |

### PRPs are proline rich peptides.

The following sources of materials were used in conjunction with the present invention.

### Material Source

### 1. Acylan LA 0478—DEGUSSA.GOLDSCHMIDT GmbH

2. Vitis venifera—Cosmetic Laboratory. AVA S.C., Warsaw, Pl.
3. Soybean oil—Cosmetic Laboratory. AVA S.C., Warsaw, Pl.

### 4. Abil 350—DEGUSSA.GOLDSCHMIDT GmbH

5. Paraffin oil—Cosmetic Laboratory. AVA S.C., Warsaw, Pl.

### 6. C.L.A.—BioOriginal

7. Vit. E—Cosmetic Laboratory. AVA S.C., Warsaw, Pl.

### 8. Lactoferrin—Tatua Nutritionals

### 9. Hippohae Ramn.—PharmaReview Corporation

### 10. Flax oil—BioOriginal

11. PRP—peptides (low molecular whey peptides) Uniwersytet Wroclawski, Instytut Biochemii I Biologii Molekulamej—Wroclaw. Pl.

### 12. Anthocyanes—Agropharm S.A.

13. Glycerin—Cosmetic Laboratory. AVA S.C., Warsaw, Pl.
14. Methyl-paraben—Cosmetic Lab. AVA S.C., Warsaw, Pl.
15. Propyl-paraben—Cosmetic Lab. AVA S.C., Warsaw, Pl.
16. 20% citric acid—Cosmetic Laboratory. AVA S.C., Warsaw, Pl.

### 17. Water added to 100%

The following sources of materials were used in conjunction with the present invention.

### Material Source

### 1. Tego® care 450—DEGUSSA.GOLDSCHMIDT GmbH

### 2. Tego® M—DEGUSSA.GOLDSCHMIDT GmbH

### 3. Tego® Alkohol 1618—DEGUSSA.GOLDSCHMIDT GmbH

### 4. Tegosoft® OS—DEGUSSA.GOLDSCHMIDT GmbH

### 5. Acylan LA 0478—DEGUSSA.GOLDSCHMIDT GmbH

6. Vitis venifera—Cosmetic Laboratory. AVA S.C., Warsaw, Pl.
7. Soybean oil—Cosmetic Laboratory. AVA S.C., Warsaw, Pl.

### 8. Abil 350—DEGUSSA.GOLDSCHMIDT GmbH

9. Paraffin oil—Cosmetic Laboratory. AVA S.C., Warsaw, Pl.

### 10. C.L.A.—BioOriginal

11. Vit. E—Cosmetic Laboratory. AVA S.C., Warsaw, Pl.

### 12. Lactoferrin—Tatua Nutritionals

### 13. Hippohae Ramn.—PharmaReview Corporation

### 14. Flax oil—BioOriginal

15. PRP—peptides (low molecular whey peptides) Uniwersytet Wroclawski, Instytut Biochemii I Biologii Molekulamej—Wroclaw. Pl.

### 16. Anthocyanes—Agropharm S.A.

17. Glycerin—Cosmetic Laboratory. AVA S.C., Warsaw, Pl.

### 18. Oxynex K liquid—Merck

### 19. Geogard® ultra—Lonza Inc.

20. Methyl-paraben—Cosmetic Lab. AVA S.C., Warsaw, Pl.
21. Propyl-paraben—Cosmetic Lab. AVA S.C., Warsaw, Pl.
22.20% citric acid—Cosmetic Laboratory. AVA S.C., Warsaw, Pl.

### 23. Water added to 100%

Yet another version of the composition of the present invention includes:

### Biovien® 1-7 Creams—Ingredients.

### 1. Tego® care 450

### 2. Tego® M

### 3. Tego® Alcohol 1618

### 4. Tegosoft® OS

### 5. Acylan LA 0478

6. Vitis venifera

### 7. Soyabean oil

### 8. Abil 350

### 9. Paraffin oil

### 10. ZOLA 1-powder

### 11. ZOLA 2-Vitamins—Oily consistency.

### 12. ZOLA 3—oily consistency Proprietary blend.

### 13. ZOLA 4—oily consistency

### 14. ZOLA 5—powder

### 15. ZOLA 6—powder

### 16. Glycerin

### 17. Oxynex K liquid

### 18. Geogard ultra

### 19. Methyl—paraben

### 20. Propyl—paraben

21. 20% citric acid
22. Vit. E

### 23. Water add to 100%.

Compounds written in red are essential for mass formation, in dark blue are biologically active elements delivered in to Biovien® cream, body lotion and dietary supplements in reverse pyramide form example see below:

| Compound: | B ®-1 | B ®-2 | B ®-3 | B ®-4 | B ®-5 | B ®-6 | B ®-7 |
|---|---|---|---|---|---|---|---|
| 1. Lactoferrin | x | x | x | x | x | x | x |
| 2. C.L.A. | 0 | x | x | x | x | x | x |
| 3. Hippohae r. | 0 | 0 | x | x | x | x | x |
| 4. Flax oil | 0 | 0 | 0 | x | x | x | x |
| 5. PRP | 0 | 0 | 0 | 0 | x | x | x |
| 6. Antocyanes | 0 | 0 | 0 | 0 | 0 | 0 | x |
| 7 Vit E. | 0 | x | x | x | x | x | x |

No 7.0 serve as preservative to remaining 2–7 compounds.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, MB, BBC, AAABCCCC, CBBAAA, CABABB, and so forth. The skilled artisan will understand that typically there is no limit on the number of items or terms in any combination, unless otherwise apparent from the context.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

### REFERENCES

1. Kehrer J P., Smith C V. 1994. Free radicals in biology: Sources, reactivities, and roles in the etiology of human diseases. 1994. The Oxidants and Antioxidants, Academic Press, 25-62.
2. Georgiades J. A., 2007. A new approach to cosmetology. Part 1. Clinical study evaluating Biovien products. Submitted.
3. Kruzel, M. L., Zimecki M. 2002. Lactoferrin and Immunologic Dissonance: Clinical Implication. Arch. Immunol. et Therapiae Experimentalis, 50, 399-410.
4. Artym, J., Zimecki, M., Kuryszko, J., Kruzel, M. L. 2005. Lactoferri Accelerates Reconstitution of the Humoral and Cellular Immune Response During Chemotherapy-induced Immunosuppresion and Bone Marrow transplant in Mice. Steam Cell and Development 14: 548-555.
5. Georgiades, J., F. Coraggio, V. Coto, C. A. Galeota, and J. Crowell-Nasti. 1969. Studies on human unspecific resistance mechanisms in the course of viral infections. I. Some biological properties of polyoma-type inhibitors isolated from human serum. Boll. Inst. Seroter. Milanese, 48: 629-635.
6. Georgiades, J., M. Zembala, F. Coraggio, V. Coto, C. A. Galeota, and J. Crowell-Nasti. 1969. Ibid. II. The level of influenza B and polyoma virus inhibitors in sera of patients suffering from different viral diseases. Boll. Inst. Sieroter. Milanese, 48: 636-645.
7. Georgiades J., M. Zembala, F. Coraggio, V. F Coto, C. Galeota. 1970. Studies on human unspecific resistance mechanisms. III. Purifications and analysis of human serum thermostable inhibitors. Boll. Inst. Sierot. Milanese, 49.
8. Georgiades, J., M. Zembala, F. Coraggio, V. Cota, C. Galeota, and J. Crowell-Nasti. 1970. IV. The mechanism of action of thermostable inhibitors of the human serum. Boll. Inst. Sierot. Milanese, 49: 42-47.
9. Georgiades, J., F. Coraggio, L. Manguso, J. Crowell-Nasti, C. Galeota, and V. Coto. 1970. V. Studies on aggregation and dissociation of polyoma and myxovirus thermostable inhibitors. Boll. Inst. Sierot. Milanese, 49: 48-53.
10. Baron, S. 1963. Mechanism of recovery from viral infection. In: Smith K M; Laufer A M (eds). Advances in Virus research. New York: Academic Press. Inc. 10:39-64, 1963.
11. Dianzani F., Baron S., Nonspecific Defenses in: Medical Microbiology, Fourth edition S. Baron (ed). The University of Texas, Medical Branch at Galveston, Tex. 1984. pp. 631-640.
12. Baron, S., J. E. Blalock, F. Dianzani, R. Fleishmann, Jr., J. A. Georgiades, H. M. Johnson, and J. Stanton. 1979. Interferon: Antiviral, immunoregulatory and anti cellular activities. In: Virus-Lymphocytes Interactions; Implication for Disease (M. R. Proffitt, ed.) Elsever North Holland Biomedical Press, New York. PBL Interferon Source 2007., 131 Ethel road West, suite 6. Piscateway, N.Y., 08854 USA.
13. Mutchucumaran, G., Kotenko, S., Donelly, R., Ihle, J. N. Pestka, S. 1997. Jak-Stat signal transmission pathway through the eyes of cytokine. J. Biol. Chem. 272, 4993-4999
14. Georgiades, J. A. 2006 Nowa metoda kontroli wolnych rodników. Przeciwdzialanie starzeniu. Czasopismo Aptekarskie. 148,(4), 30-36.
15. Georgiades, J. A., 2006. Mechanizmy odpomości u ludzi I wolne rodniki. Med. Estetyczna i Przeciwstarzeniowa, 5.(3), 110-119.
16. Hughes, T. K., S. Baron. 1987. A large component og the antiviral activity of interferon-γ may be due to its induction of Interferon-α. J. Biol. Regul. Homeostatic Agents. 1.29.
17. Hughes, T. K., S. Baron. 1987. Do the IFN's act singly or in combinations? J. Interferon Res. 7:603.
18. Fleishmann, W. R., Jr. Georgiades, J. A., Osborn, L. C. and Johnson, H. M. 1979. Potentiation of interferon activity by mixed preparation of fibroblast and immune interferon. Inf. Immunity, 26 (1) 26-41.
19. Blalock J. E., S. Baron. 1971. Interferon induced transfer of the antiviral resistance between animal cells. Nature. 269: 422-25.
20. Blalock J. E., Georgiades, J. A., Johnson H. M. 1979. Immune type of interferon induced transfer of viral resistance. J. Immunol. 122: 1018-1021.
21. Georgiades, J. A. 1998. Oral cavity gate of an entry into the Communication Network. Biotherapy. 11: 39-51.
22. Boldogh, I., Hughes Jr. T. K., Georgiades, J. A., Stanton, G. J. 2001. Antioxidant effect of Colostrinin and its component peptides. Psychogeriatric Annals, 4, 57-65.
23. Boldogh, I., Liebenthal, D., Hughes, Jr. T. K., Juelich, T. I., Georgiades, J. A., Kruzel, M. L., Stanton G. J. 2003. Modulation of 4HNE-mediated Signaling by Proline Rich peptides from Ovine colostrums. J. Molecul. Neurosci. 20: 125-134.

What is claimed is:

1. A medicinal composition to prevent or control the effects of free radicals on skin comprising:
a composition comprising a metalloprotein and fatty acids in an amount sufficient to improve the appearance of skin; and optionally comprising one or more of the following:
(a) conjugated linoleic acids and vitamin E;
(b) conjugated linoleic acids, plant-derived lipids, Hippohae rhamnoides L. berry extract (“oblepicha”) and vitamin E;
(c) conjugated linoleic acids, plant-derived lipid, Hippohae rhamnoides L. berry extract, omega fatty acids and vitamin E;
(d) conjugated linoleic acids, plant-derived lipid, Hippohae rhamnoides L. berry extract, omega fatty acids, low molecular weight whey protein extract and vitamin E;
(e) conjugated linoleic acids, plant-derived lipid, Hippohae rhamnoides L. berry extract, omega fatty acids, low molecular weight whey protein extract and vitamin E; and
(f) conjugated linoleic acids, plant-derived lipid, Hippohae rhamnoides L. berry extract, omega fatty acids, low molecular weight whey protein extract, vitamin E and Antocyanes,
wherein the addition of (a) to (f) is dependent on the extent of free-radical damage to the skin, wherein (a) is used to treat a lower level of skin damage and (f) is used to treat the highest levels of free-radical damage to skin, with (b) to (e) added progressively as skin damage increases, respectively.

2. The composition of claim 1, further comprising an equivalent oral equivalent of the composition.

3. The composition of claim 1, further comprising an oral equivalent of the composition and a cream equivalent of the composition.

4. The composition of claim 1, wherein the improve of the appearance of skin is measured by decreased pH, inflammation, comedon, acne, hyperkeratosis, enlargement of sebum vessels, pores enlargement, piling centers, skin discoloration or scars.

5. The composition of claim 1, further comprising one or more anti bacterial agents.

6. The composition of claim 1, wherein the metalloprotein comprises ferritin or lactoferrin.

7. The composition of claim 1, wherein the composition is non-toxic.

8. The composition of claim 1, wherein the composition is non-allergenic.

9. The composition of claim 1, further comprising one or more anti bacterial agents.

10. The composition of claim 1, wherein the low molecular weight whey protein extract comprises small molecular weight peptides from whey fraction from 3 to 14 kilodaltons.

11. A method for preventing and treating the effects of free-radicals using a medicinal progression of anti-oxy-vitamins comprising:
treating the skin of a subject based on the extent of free-radical damage to the skin, wherein a first, second, third, fourth, fifth, sixth or seventh composition is selected based on an increased level of severity of the free-radical damage to the skin in ascending order, respectively, the compositions comprising:
a first composition comprising lactoferrin and fatty acids in an amount sufficient to decrease oily secretions from skin;
a second composition comprising a metalloprotein, fatty acids, conjugated linoleic acids and vitamin E;
a third composition comprising a metalloprotein, fatty acids, conjugated linoleic acids, Hippohae rhamnoides L. berry extract (“oblepicha”) and vitamin E;
a fourth composition comprising a metalloprotein, fatty acids, Hippohae rhamnoides L. berry extract, omega fatty acid and vitamin E;
a fifth composition comprising a metalloprotein, conjugated linoleic acids, Hippohae rhamnoides L. berry extract, omega fatty acids, low molecular weight whey protein extract and vitamin E;
a sixth composition comprising a metalloprotein, conjugated linoleic acids, Hippohae rhamnoides L. berry extract, omega fatty acids, low molecular weight whey protein extract and vitamin E; and
a seventh composition comprising a metalloprotein, conjugated linoleic acids, Hippohae rhamnoides L. berry extract, omega fatty acids, low molecular weight whey protein extract, vitamin E and antocyanes, wherein a patient uses one or more of the first to seventh serums based on the condition of their skin.

12. The method of claim 11, wherein the low molecular weight whey protein extract comprises small molecular weight peptides from whey fraction from 3 to 14 kilodaltons.

13. The method of claim 11, wherein the effects of free-radicals on skin is measured by at least one of decreased pH, inflammation, comedon, acne, hyperkeratosis, enlargement of sebum vessels, pores enlargement, piling centers, skin discoloration or scars.

14. A composition comprising a metallo-protein, vitamin E, pro-vitamin A, vitamin H, vitamin B, vitamin C, vitamin D, vitamin E, low molecular weight whey protein extract and antocyanes, wherein the composition had a greater free radical scavenging effect than the individual agents alone.

15. The composition of claim 14, further providing comprising an equivalent oral equivalent of the composition.

16. The composition of claim 14, further comprising an oral equivalent of the composition and a cream equivalent of the composition.

17. The composition of claim 14, wherein the metalloprotein comprises lactoferrin.

18. The composition of claim 14, further comprising one or more anti bacterial agents.

19. The composition of claim 14, wherein the composition is non-toxic.

20. The composition of claim 14, wherein the composition is non-allergenic.

21. A composition for reducing the pH of skin comprising:
Lactoferrin and conjugated linoleic acids adapted for topical administration, each of which is provided in an amount sufficient and to lower the pH of skin.

22. The composition of claim 21, further comprising at least one of a Hippohae rhamnoides L. berry extract, Flax oil, anthocynaes and Vitamin E.

23. A medicinal progression of anti-oxidants comprising:
a first composition comprising lactoferrin and fatty acids;
a second composition comprising lactoferrin, fatty acids, conjugated linoleic acids and vitamin E;
a third composition comprising lactoferrin, fatty acids, conjugated linoleic acids, Hippohae rhamnoides L. berry extract (“oblepicha”) and vitamin E;
a fourth composition comprising lactoferrin, fatty acids, Hippohae rhamnoides L. berry extract, omega fatty acid and vitamin E;
a fifth composition comprising lactoferrin, conjugated linoleic acids, Hippohae rhamnoides L. berry extract, omega fatty acids, low molecular weight whey protein extract and vitamin E;
a sixth composition comprising lactoferrin, conjugated linoleic acids, Hippohae rhamnoides L. berry extract, omega fatty acids, low molecular weight whey protein extract and vitamin E; and
a seventh composition comprising lactoferrin, conjugated linoleic acids, Hippohae rhamnoides L. berry extract, omega fatty acids, low molecular weight whey protein extract, vitamin E and antocyanes, wherein a patient uses one or more of the first to seventh serums based on the condition of their skin.

24. A method of treating one or more skin conditions comprising:
evaluating one or more skin conditions in a patient selected from at least one of decreased pH, inflammation, comedon, acne, hyperkeratosis, enlargement of sebum vessels, pores enlargement, piling centers, skin discoloration or scars; and
selecting one or more of the following serums that best treats the one or more skin conditions selected from:
a first composition comprising lactoferrin and fatty acids;
a second composition comprising lactoferrin, fatty acids, conjugated linoleic acids and vitamin E;
a third composition comprising lactoferrin, fatty acids, conjugated linoleic acids, Hippohae rhamnoides L. berry extract (“oblepicha”) and vitamin E;
a fourth serum comprising lactoferrin, fatty acids, Hippohae rhamnoides L. berry extract, omega fatty acid and vitamin E;
a fifth composition comprising lactoferrin, conjugated linoleic acids, Hippohae rhamnoides L. berry extract, omega fatty acids, low molecular weight whey protein extract and vitamin E;
a sixth composition comprising lactoferrin, conjugated linoleic acids, Hippohae rhamnoides L. berry extract, omega fatty acids, low molecular weight whey protein extract and vitamin E; and
a seventh composition comprising lactoferrin, conjugated linoleic acids, Hippohae rhamnoides L. berry extract, omega fatty acids, low molecular weight whey protein extract, vitamin E and antocyanes, wherein a patient uses one or more of the first to seventh serums based on the condition of their skin.

25. The method of claim 24, wherein the patient is further provided with an equivalent oral equivalent of the composition.

26. The method of claim 24, wherein the patient is further provided with an oral equivalent of the composition and a cream equivalent of the composition.

27. The method of claim 24, wherein the serum further comprises one or more anti bacterial agents.

28. The method of claim 24, wherein the serum is non-toxic.

29. The method of claim 24, wherein the serum is non-allergenic.

30. A method reducing the pH of skin comprising:
identifying a subject in need of skin pH correction; and
applying to the subject a composition comprising Lactoferrin and conjugated linoleic acids adapted for topical administration, each of which is provided in an amount sufficient and to lower the pH of skin.

31. The method of claim 30, wherein the composition further comprises at least one of a Hippohae rhamnoides L. berry extract, Flax oil, anthocynaes and Vitamin E.

32. The method of claim 30, wherein the subject is further provided with an equivalent oral equivalent of the composition.

33. The method of claim 30, wherein the subject is further provided with an oral equivalent of the composition and a cream equivalent of the composition.

34. The method of claim 30, wherein the composition further comprises one or more anti bacterial agents.

35. The method of claim 30, wherein the composition is non-toxic.

36. The method of claim 30, wherein the composition is non-allergenic.

37. The method of claim 30, wherein the composition comprises lactoferrin, fatty acids, conjugated linoleic acids, Hippohae rhamnoides L. berry extract (“oblepicha”) controls aging as measured by at least one of reduced decreased pH, inflammation, comedon, acne, hyperkeratosis, enlargement of sebum vessels, pores enlargement, piling centers, skin discoloration or scars.

38. A method reducing the pH of skin comprising:
identifying the needs of a subject for skin treatment by evaluating at least one of decreased pH, inflammation, comedon, acne, hyperkeratosis, enlargement of sebum vessels, pores enlargement, piling centers, skin discoloration or scars;
matching the needs of the subject with an anti-oxy-vitamin composition adapted for topical administration comprising Lactoferrin, conjugated linoleic acids and one or more of the following agents: Hippohae rhamnoides L. berry extract, Flax oil, anthocynaes and Vitamin E; and
applying to the subject the composition in an amount sufficient and to treat the at least one of inflammation, comedon, acne, hyperkeratosis, enlargement of sebum vessels, pores enlargement, piling centers, skin discoloration or scars.

39. The method of claim 38, wherein the composition slows down the appearance of aging as measured by at least one of reduced skin wrinkles, increases in skin elasticity, reduced skin discoloration, reduced scar formation, reduced sebum production, increased hydration effect and decreased skin pH.

40. The method of claim 38, further comprising providing the patient concomitantly with an equivalent of the composition for skin treatment in a form adapted for oral administration.

41. A method for reversing the damage to skin caused by free-radicals comprising:
applying to the skin of a subject with skin damage with an effective amount of an anti-oxy-vitamin composition adapted for topical administration comprising Lactoferrin, conjugated linoleic acids and one or more of the following agents: Hippohae rhamnoides L. berry extract, Flax oil, anthocynaes and Vitamin E, wherein the skin damage comprises at least one of decreased pH, inflammation, comedon, acne, hyperkeratosis, enlargement of sebum vessels, pores enlargement, piling centers, skin discoloration or scars.
